(19) United States
(12) Patent Application Publication
(10) Pub. No.: US 2021/0378851 A1
(43) Pub. Date: Dec. 9, 2021

(54) System and Method for Treating Critical Limb Ischemia (CLI) via the Superficial Femoral Arteries (SFA)

(71) Applicant: Gregory Sullivan, Quincy, MA (US)

(72) Inventor: Gregory Sullivan, Quincy, MA (US)

(21) Appl. No.: 17/339,565

(22) Filed: Jun. 4, 2021

Related U.S. Application Data
(60) Provisional application No. 63/034,623, filed on Jun. 4, 2020.

(51) Int. Cl. A61F 2/966; A61F 2/958

(52) U.S. Cl. CPC A61F 2/966; A61F 2/958

(57) ABSTRACT

Systems, methods and accompanying apparatuses to treat critical limb ischemia through the superficial femoral arteries utilizing a specifically designed delivery package for entry proximal to area above the ankle with the capability to antegrade access via PT or AT into ankle and foot and retrograde access via pedal or plantar arch into the dorsalis pedis artery (DP).

### CROSS REFERENCE TO RELATED APPLICATION

This application claims priority to and takes the benefit of Provisional Patent Application Ser. No. 63/034,623 filed on Jun. 4, 2020, the contents of which are herein incorporated by reference

### BACKGROUND OF THE INVENTION

### Field of the Invention

The instant system relates generally to medical devices and systems, including surgical and medical delivery systems. More particularly, the instant system relates to stents, catheters, and sheaths and procedures for treating Critical Limb Ischemia and other lower limb maladies.

### Description of the Related Art

Within the art, currently disposed stent devices include, but are not limited to, elongated device used in many capacities, including but not limited to support an intraluminal wall. Stenosis is an abnormal narrowing in a blood vessel or other tubular organ or structure. This vessel narrowing prevents the valve from opening fully, which obstructs blood flow from the heart and onward to the rest of the body.

There concurrently exist a wide variety of stents used for different purposes depending on the type of narrowing of a vessel in the body required. As used herein, the term “stent” is a shorthand reference referring to the wide varieties of stents, both covered and uncovered.

Stents are typically implanted within the vascular system to reinforce collapsing, partially occluded, weakened or under dilated sections of vessel and valves. Stents have also been successfully implanted in urinary tracts and bile ducts to reinforce those body vessels. This invention is applicable in all of these situations.

In general, the typical procedure for implanting a self-expanding stent is to first open the region of the vessel with a balloon catheter and then place the stent in a position bridging the weakened the portion of the vessel. Positioning of the stent may be followed by the technique known as the “Swiss Kiss” in which a separate balloon catheter is positioned within the stent and expanded to radially expand the stent for implantation.

Currently there are no favorable stents on the market to meet needs for below the ankle due to ankle strap crush effect and the ankle area is susceptible to high stress, torsion, extension, and body weight.

Acute arterial occlusion is a serious problem. In one manner, this condition occurs when blood flow in a leg artery is suddenly stopped. If blood flow to your toe, foot, or leg is completely blocked, the tissue begins to die. This is called gangrene. If this happens, medical care is needed right away to restore blood flow and possibly save a patient's foot and/or limb.

Just as in any such condition serious health condition, there are numerous causes of acute arterial occlusion. Patients are more likely to come down with this condition if a patient possesses an antecedent case of peripheral arterial disease (PAD). With PAD, leg arteries are narrowed, thus reducing blood flow to the legs and feet of the patient.

In order to diagnose acute arterial occlusion, the tests may include the ankle-brachial index (ABI) wherein the blood pressure in your ankle is compared to the blood pressure in your arm, often utilizing a duplex ultrasound. Sound waves are utilized to create images of blood flow in your legs; and an arteriography. X-ray dye (contrast medium) is injected into the artery. This is done using a thin, flexible tube (catheter) wherein the dye makes blood vessels show up more clearly on X-rays.

There are many manners in which to treat acute arterial occlusion and possible treatments for acute arterial occlusion may include dissolving or removing a blood clot wherein a tube (catheter) may be placed into an artery, normally in the groin to dissolve the clot. Next clot-busting medicine is then put into the tube to dissolve the clot. Or surgery may be done to remove the clot. A cut (incision) is made in the artery at the blocked area. The clot is then removed. Additionally, in the angioplasty, a procedure which embraces passing an uninflated balloon through a catheter to the narrowed part of the artery and inflating the balloon to widen the artery and finally deflating and removing the balloon.

An additional treatment methodology may utilize stenting which may be utilized in conjunction with angioplasty and wherein a mesh like member, often referred to as a stent, may be inserted within a spread artery to retain the artery in open position. In many applications, the stent may be delivered and lodged utilizing a catheter system.

A further treatment methodology may include Endarterectomy, a process wherein an incision is made in the artery at the blocked area in order to remove the blockage from the annular area and artery walls. Moreover, drastic and life saving measure may be Peripheral bypass surgery, wherein a graft, be it manmade or natural is installed in order to bypass the blocked area of an artery.

One of the resultant conditions if treatment does not occur or is not successful is Critical Limb Ischemia (CLI) which may be the most severe manifestation of PAD resulting in ulcers and gangrene. Critical limb ischemia is the advanced stage of peripheral artery disease (PAD), which results from a progressive thickening of an arteries lining (caused by a buildup of plaque). This buildup of plaque, also known as atherosclerosis, narrows or blocks blood flow, reducing circulation of blood to the legs, feet or hands and markedly reduces blood flow to the extremities (hands, feet and legs) and has progressed to the point of severe pain and even skin ulcers or sores. CLI often requires comprehensive treatment by a vascular surgeon or vascular specialist. This condition will not improve on its own.

The pain caused by CLI can wake up an individual while in bed and sound asleep. This pain, also called “rest pain,” is often in the leg and can be relieved temporarily by hanging the leg over the bed or getting up to walk around. Nearly two (2) million people in the US and Europe suffer from CLI and CLI results in nearly 230,000 amputations every year.

Normally, indications for revascularization are wherein a patient has the following: a non-healing foot ulcer, a non-healing foot wound and gangrenous foot changes, in and Skin Perfusion Pressure (SPP) less than 30 mm Hg Revascularization is necessary for healing.

Concurrently, when dealing with patients in the throes of critical limb ischemia and exhibiting those issue delineated above, the treating medical team must decide which patient is a candidate for limb salvage, and this has always been an open-ended question due to the limitations of the treatment methods, as well as the restrictive nature of access to the pertinent arteries and veins within the lower extremities. The advancements herein qualify any patient, regardless of anatomy, in whom the only alternative is deemed to be amputation.

Peripheral arterial disease (PAD) is a common manifestation of atherosclerosis affecting 5 million adults in the United States, with an age-adjusted prevalence of 4% to 15% and increasing up to 30% with age and the presence of cardiovascular risk factors. In this article we focus on lower extremity PAD and specifically on the superficial femoral and proximal popliteal artery (SFPA), which are the most common anatomic locations of lower extremity atherosclerosis.

Chronic lower extremity ischemia, also known as peripheral artery disease (PAD), is a common condition managed by vascular specialists. The primary etiology is atherosclerosis. Atherosclerotic stenosis or occlusion of the peripheral arterial tree results in arterial insufficiency and end-organ (limb) ischemia. PAD is a major contributor to morbidity, reduced quality of life (QOL), and mortality in an increasing elderly demographic in the Western world.

As the population ages, it is anticipated that the prevalence of peripheral vascular disease will increase. Within the past decade there has been an unprecedented evolution of the endovascular technologies and vital improvements are expected in the next decade. Percutaneous procedures will continue to replace open surgery. The chief challenge in the management of peripheral arterial disease would be retooling of the health system to focus on identifying patients with PAD and taking the enormous opportunity and responsibility to refine and aggressively manage the atherosclerotic risk factors in these patients.

The superficial femoral artery is a continuation of the common femoral artery at the point where the profunda femoris branches. It is the main artery of the lower limb and is, therefore, critical in the supply of oxygenated blood to the leg. Over the past decade there has been a remarkable advancement in the endovascular treatment of lower extremity PAD with the introduction of new interventional techniques and devices, and specifically on the superficial femoral and proximal popliteal artery (SFPA), which are the most common anatomic locations of lower extremity atherosclerosis.

Currently, endovascular treatment of SFPA disease is indicated for individuals with significant disability due to intermittent claudication or critical limb ischemia when clinical features suggest a reasonable likelihood of symptomatic improvement with endovascular intervention, there has been an inadequate response to exercise or pharmacological therapy and when there is a favorable risk-benefit ratio.

Endovascular surgery is an innovative, less invasive procedure used to treat problems affecting the blood vessels, such as an aneurysm, which is a swelling or “ballooning” of the blood vessel. The surgery involves making a small incision near each hip to access the blood vessels. An endovascular graft, which is a special fabric tube device framed with stainless steel self-expanding stents, is inserted through the arteries in a catheter, a long, narrow flexible tube, and positioned inside the aorta. Once in place, the graft expands and seals off the aneurysm, preventing blood from flowing into the aneurysm. The graft remains in the aorta permanently.

Apart from the clinical and angiographic criteria for selection of patients for endovascular treatment, for stenoses of 50-75% diameter by angiography, intravascular translesional pressure gradients have been recommended to determine whether these lesions are hemodynamically significant and to predict patient improvement after revascularization. Although there is no consensus on the diagnostic translesional pressure gradient criteria, the most widely accepted criteria utilize a mean gradient of 10 mmHg before or after vasodilators; or a mean gradient of 5 mmHg and peak systolic gradient of 10, 15 or 20 mmHg; or 15% peak systolic pressure gradient after administration of a vasodilator. The TASC II consensus emphasizes more anatomic criteria and recommends endovascular revascularization for type A lesions and surgery for type D lesions, whereas endovascular treatment is preferred for type B lesions and surgery for good-risk patients with type C lesions.

For some time, surgical revascularization has been the main form of treatment and surgical treatment of lower extremity ischemia is indicated for patients with claudication and significant functional disability or critical limb ischemia, after failure of conservative or endovascular therapy, who have a reasonable likelihood of symptomatic improvement, favorable limb arterial anatomy and low cardiovascular risk for surgical revascularization. With the evolution of endovascular technology and interventional techniques as well as their equivalent efficacy, lower cost and lower peri-procedural risk, surgery has become a second-line revascularization option and is currently recommended only for TASC D lesions.

Furthermore, as surgery should be avoided in patients younger than 50 years old, since they have a more virulent form of atherosclerosis and subsequently a higher frequency of graft failure requiring revisions and replacement, minimally invasive surgery is plainly the better choice. Minimally invasive surgery is a surgery minimizing surgical incisions to reduce trauma to the body. This type of surgery is usually performed using thin-needles and an endoscope to visually guide the surgery. Minimally invasive surgery doctors use a variety of techniques to operate with less damage to the body than with open surgery. In general, minimally invasive surgery is associated with less pain, a shorter period in the hospital and fewer complications.

Normally, once the decision to proceed with surgical intervention is undertaken, the type of revascularization should be elected based on different variables, such as location and severity of disease, anatomy, general medical condition, prior revascularization attempts and the desired outcome. As a general rule, in patients with combined inflow and outflow disease, inflow problems are corrected first, since improvement of the inflow may diminish the symptoms of claudication and reduce the likelihood of distal graft thrombosis from low flow. In the case of superficial femoral and proximal popliteal artery (“SFPA”) disease, two major factors that can modify the result of the procedure are the type of the conduit and the site of the distal anastomosis.

The superior rates of immediate and long-term patency rates favor autogenous vein grafts as opposed to prosthetic conduits for both above—or below-the-knee bypasses. In some studies, the 5-year patency rates of femoropopliteal bypass grafts are reported as 80% for vein grafts, 75% for above-the-knee synthetic grafts and 65% for below-the-knee synthetic grafts. Patients undergoing surgical bypass for lower extremity ischemia should be entered into a clinical surveillance program that consists of interval history and vascular exam as well as measurement of resting and, if possible, post-exercise ABIs and duplex imaging of the entire length of the graft with measurements of peak systolic velocities and calculation of the velocity ratios across all lesions in the immediate postoperative period and at regular intervals (usually every 6 months) for at least 2 years.

Major amputation in patients with critical or acute limb ischemia should be reserved only when the limb is unsalvageable, i.e., when there is overwhelming infection that threatens the patient's life, extensive necrosis or refractory ischemic rest pain.

Currently, a wide variety of established and evolving endovascular techniques to treat PAD including percutaneous transluminal angioplasty (PTA) with balloon dilation, stents, endografts, atherectomy, laser, cutting balloons, drug-coated balloon angioplasty, cryoplasty, percutaneous thrombectomy and brachytherapy exist. Endovascular technological advances have made a minimally invasive percutaneous approach the treatment of choice in the initial management of the majority of symptomatic patients over the traditional surgical approach. However, the presence of severe vascular calcification, particularly in the infrainguinal vasculature, presents a significant procedural challenge to current endovascular strategies. Due to a lack of large randomized, prospective trials with independent core laboratory adjudication of device-related acute and late events, operators have used different approaches to treat femoropopliteal or infrapopliteal disease. Percutaneous transluminal angioplasty (PTA) or balloon angioplasty has been traditionally used for treatment of focal lesions.

However, early elastic recoil, frequent dissections, and poor primary and secondary patency rates for long lesions, with 40-50% of cases requiring bail-out stenting, limit balloon angioplasty of “severely” calcified lesions, despite the high procedural success rates. Although the use of the last generation self-expanding nitinol stents may be an effective treatment for focal lesions, with high acute procedural success rates, restenosis rates can be as high as 10-40% at six to 24 months, and stent fractures may occur at sites of excessive movement and flexion.

Furthermore, the presence of rigid calcified plaques may result in incomplete stent expansion and significant residual stenosis. Lack of effective therapy for restenosis has led many interventional cardiologists to seek alternative treatment strategies, such as plaque modification by means of debulking, using an atherectomy device.

Atherectomy is a procedure performed to remove or “debulk” the atherosclerotic plaque from diseased arteries. It is usually combined with low-pressure balloon angioplasty with the goal of minimizing plaque shift while avoiding stent placement. In densely calcified vessels, atherectomy has been used to better “prepare” the vessel prior to stenting in order to prevent incomplete and/or eccentric stent expansion. There are different atherectomy devices designed to cut, shave, sand, or vaporize atherosclerotic or calcified plaques, and they have slightly different indications that depend on the lesion characteristics. Four different methods of atherectomy have been utilized for treatment of femoropopliteal or small-vessel infrapopliteal disease: plaque excision (directional) atherectomy, rotational atherectomy/aspiration, laser atheroablation, and orbital atherectomy.

Directional or extractional atherectomy devices utilize carbide rotating cutter disks that resect and remove the atherosclerotic plaque. These devices have the advantage of avoiding barotrauma, which may decrease the risk of neointimal hyperplasia and dissection. Distal embolization remains a concern with these devices, given that these devices require retrieval of removed plaque, and the use of distal protection devices may be needed, particularly in cases of heavily calcified lesions.

The SilverHawk® and TurboHawk® (Covidien/Medtronic) plaque excision systems are the two U.S. Food and Drug Administration (FDA)-approved directional atherectomy devices in use today, with the HawkOne® system recently being FDA cleared as well. The SilverHawk® and TurboHawk® devices come in various sizes to enable atherectomy in vessels ranging from a diameter of 1.5 mm to 7 mm. The SilverHawk® plaque excision system is a forward-cutting, directional atherectomy device that consists of a rotating blade inside a tubular housing with a collection area. The TurboHawk® system is similar to the SilverHawk® except with a different number of inner blades, allowing for a larger luminal gain.

While SilverHawk® has one inner blade, TurboHawk® has four contoured blades, thus favoring use in highly calcified lesions and achieving more plaque removal per pass. As the name implies, the new HawkOne® system is intended to simplify device selection, allowing treatment of lesions with different morphologies (including severe calcification) with one device. The Determination of Effectiveness of SilverHawk® Peripheral Plaque Excision [SilverHawk® Device] for the Treatment of Infrainguinal Vessels/Lower Extremities (DEFINITIVE LE) study is the largest study to evaluate directional atherectomy, with enrollment of 800 patients worldwide with both claudication and/or CLI across 50 sites in the U.S. and Europe. The device success was reported at 89%, with a post-atherectomy bail-out stenting rate of 3.2%. Rates of distal embolization, dissection, and perforation were 3.8%, 2.3%, and 5.3%, respectively. At 12 months, primary patency rate in claudicants was 78%, whereas the rate of freedom from major unplanned amputation of the target limb at 12 months in CLI subjects was 95%.

Rotational atherectomy is currently available as the Jetstream Atherectomy (currently Boston Scientific, previously Pathway Medical Technologies, Inc.), and the Phoenix atherectomy catheter (AtheroMed®). The Boston Scientific/Pathway Jetstream® Atherectomy System is a single-use catheter with a reusable, compact console power source and a front-cutting tip that allows it to go through severely stenotic lesions without predilation. It is the only atherectomy device to offer continuous aspiration and active removal of atherosclerotic debris and thrombus.

Thus, this device may be particularly useful in lesions of mixed morphologies, particularly those with presence of thrombus (e.g., acute or subacute occlusions). In a multicenter study of 172 patients, Jetstream use had a 99% device success, and six-month and 12-month clinically-driven, target-lesion revascularization rates of 15% and 26%, respectively; with a one-year restenosis rate of 38% based on duplex imaging.⁵ The Phoenix atherectomy device is still under investigation and the Endovascular Atherectomy Safety and Effectiveness (EASE) study is currently evaluating the safety and short-term efficacy of the Phoenix device.

Excimer Laser atherectomy (Spectranetics) uses the high-energy, monochromatic light beam to alter or dissolve (vaporize) the plaque without damaging the surrounding tissue. These devices include a Turbo Elite ablation catheter as well as a Turbo-Tandem system that combines a laser guide catheter with an excimer laser atherectomy catheter. In the Laser Angioplasty for Critical Limb Ischemia (LACI) trial, 155 critically ischemic limbs with above- or below-knee disease that were poor candidates for surgical revascularization were treated with excimer laser-assisted intervention with a limb-salvage rate of 93% at six months.

The excimer laser has an advantage of not only debulking, but also being able to penetrate the proximal fibrous cap in chronic total occlusions. Thus, it may be advantageous for utilization when the intention is to enhance crossing capability as well as further debulk the occluded vessel. Recent results from the Excimer Laser Randomized Controlled Study for Treatment of FemoropopliTEal In-Stent Restenosis (EXCITE ISR) trial of 250 patients demonstrated the safety and efficacy of excimer laser atherectomy in femoropopliteal in-stent restenosis.

Orbital atherectomy is an atherectomy device with an eccentrically-mounted crown that is being used to reduce the total atheroma burden and decrease the vessel-wall trauma, particularly in calcified vessels. Orbital atherectomy utilizes a diamond-coated tungsten crown that orbits 360 degrees eccentrically within the vessel, while employing circumferential plaque removal by differential sanding.

The currently available orbital atherectomy devices include the CSI Stealth 360 and Diamondback 360 Orbital atherectomy systems (Cardiovascular Systems, Inc). The CONFIRM registry series evaluated the use of orbital atherectomy in peripheral lesions of the lower extremities and showed that it effectively reduced the degree of stenosis from 88% to ˜10% with the use of adjunctive low-pressure balloon angioplasty. The COMPLIANCE 360° trial compared acute and 12-month results in 50 patients between orbital atherectomy plus PTA versus PTA in calcified femoropopliteal disease.¹⁰ At 12 months, freedom from TLR or restenosis was achieved in ˜80% of lesions in both groups, despite rare use of bail-out stenting after atherectomy (5.3% vs. 77.8% in PTA). The peri-procedural adverse events were seen infrequently with orbital atherectomy: perforations (0%), dissections (16%), and embolization (2.6%). Given its mechanism of action, orbital atherectomy may be particularly advantageous in severely calcified lesions, while minimizing vessel wall trauma and need for bail-out stenting. Furthermore, newer low-profile (4 French) systems allow for alternative access options, such as tibiopedal approach.

Despite different choices of atherectomy devices and advanced technologies, there have been no comparative efficacy or safety studies evaluating the four FDA-approved atherectomy devices. Atherectomy devices can reduce the burden of soft atheromatous or calcific plaque, change the vessel compliance, reduce vessel wall trauma, leading to a decrease in the need for bail-out stenting.¹¹ On the other hand, atherectomy devices carry significantly higher capital equipment-related costs, particularly when used in conjunction with distal protection filters, and lead to an increase in procedure duration and exposure to radiation. Given the availability of multiple atherectomy devices, in day-to-day clinical practice, it is important to initially obtain expertise with a single device, paying attention to patient/lesion selection and whether to utilize distal protection. The recent encouraging data from drug-coated balloons (DCB) have renewed the interest in atherectomy devices and several ongoing randomized trials are currently evaluating a strategy of combining atherectomy and DCB. Additional studies are required to identify subsets of patients benefiting from atherectomy and to establish an optimal, cost-effective therapy, which may include a combination of atherectomy and emerging technologies, such as drug-eluting stents, DCB, and possibly bioabsorbable stent platforms to ensure a more durable patency in complex lesions.

The challenge for the vascular specialist is to determine whether the nature and severity of presenting symptoms correlate with the degree of chronic arterial insufficiency present or whether alternative etiologies, such as neuropathy, inflammation, infection, lymphatic or venous disease, and repetitive trauma, are more likely responsible. Definitive diagnosis is derived from detailed historic and physical examination findings correlated with appropriately directed noninvasive vascular laboratory and adjunctive imaging studies.

Patients with PAD may present with a spectrum of symptoms ranging in severity from none to varying degrees of claudication to severe or “critical” limb ischemia. Claudication is defined as muscular pain, cramping, aching, or discomfort in the lower limb, reproducibly elicited by exercise and relieved within 10 minutes of cessation. CLI has been traditionally defined as (1) persistent, recurring ischemic rest pain requiring opiate analgesia for more than 2 weeks and (2) ankle systolic pressure less than 50 mmHg or toe systolic pressure less than 30 mmHg (or absent pedal pulse in patients with diabetes).

Ischemic rest pain typically is nocturnal, worsens with elevation, and is relieved by dependency. Pedal pulses are absent; dependent rubor, elevation pallor, and calf muscle atrophy are frequent accompaniments. CLI also includes ischemic foot ulceration and gangrene in the setting of ankle systolic pressure less than 50 to 70 mmHg or toe systolic pressure less than 40 mmHg in patients without diabetes (<50 mmHg in diabetics).

All patients with PAD require comprehensive medical management and risk factor modification. Revascularization (either open bypass or endovascular intervention) is indicated in patients who remain symptomatic and significantly limited despite adequate risk factor modification, exercise, and medical management. The primary goal of intervention, in patients with lifestyle-limiting claudication, is to improve exercise tolerance and hence QOL. Patients with rest pain, tissue loss, and gangrene are at greater risk for limb loss and cardiovascular mortality (stroke, myocardial infarction) associated with systemic atherosclerosis than those who present with claudication alone. Revascularization in the CLI cohort is focused on wound healing and functional limb salvage as well as symptomatic relief and improvement in QOL.

The vascular specialist must first determine, given the underlying disease burden, the severity of ischemic and infectious complications as well as the patient's comorbidities, functional status, and anticipated longevity. Once it is decided that revascularization will improve the patient's functional status and QOL, these same variables, in concert with anatomic assessment of the location, extent, and severity of occlusive arterial lesions will determine whether endovascular, open, or hybrid revascularization options are indicated. When bypass is selected as the preferred revascularization option, the goals of preoperative planning involve delineation of diseased arterial segment(s), identification of the most appropriate arterial inflow source, selection of the optimal bypass target for maximal outflow and target bed perfusion, and selection of the best available conduit. In practice, conduit availability is almost always a critical, rate-limiting factor because good quality, autogenous vein conduit is preferred in almost every circumstance.

Adequate preoperative planning depends on a thorough history and detailed physical examination and thus the delineation of the relevant arterial anatomy on the index limb is facilitated by high quality, noninvasive vascular laboratory studies (ankle-brachial index and toe pressure measurements). These are supplemented by arterial color duplex ultrasound imaging. Arterial duplex is extremely accurate in the assessment of iliofemoral and femoropopliteal arterial occlusive disease but less so for infrageniculate (tibial-peroneal) lesions. Duplex enables differentiation of stenosis from occlusion and determination of lesion length and degree of calcification. Cross-sectional imaging studies such as computed tomography angiography (CTA) or magnetic resonance arteriography (MRA) may add complementary information, but most experienced operators prefer the precision and resolution inherent in catheter-based, intraarterial contrast arteriography for definitive preoperative planning, especially when bypass will be required to distal calf or pedal targets.

PAD is a coronary artery disease equivalent. Therefore, preoperative risk evaluation for overall cardiovascular-related mortality represents a component of preoperative planning. In most patients with stable or minimally symptomatic coronary disease, preoperative risk-reduction efforts are best focused on optimizing medical management. Frequently, this includes statin and antiplatelet therapy, R-blockade, and optimization of hypertension management. The surgical plan should be tailored to each patient's needs based on extent of disease, conduit availability, and realistic long-term functional potential. Infrainguinal bypass may originate from the common, superficial, or deep femoral artery or the popliteal artery with a bypass target of the popliteal, tibial, or pedal/plantar arteries. The positioning, choice of incisions, and surgical techniques are dictated by type of bypass procedure deemed most appropriate under the circumstance.

Discussing some state of the art techniques, including techniques utilizing vessel exposure, the patient may be placed in a supine position and a Foley catheter is inserted. Arms may be tucked to facilitate intraoperative prebypass and completion angiography.

Regarding placement of an incision, the common femoral artery (CFA) is located on a line between the pubic tubercle and anterior iliac spine, two fingerbreadths lateral to pubic tubercle. Palpation of the inguinal ligament and femoral pulse or direct arterial visualization with duplex imaging can localize the CFA bifurcation and guide optimal incision placement. Even when pulseless due to excessive calcification or occlusive disease, the CFA may be localized by reliance on anatomic landmarks and direct palpation, recognized as a firm tubular structure positioned within the femoral sheath.

The vertical groin incision is most commonly employed to provide optimal access to the entire length of the CFA. This should be created coaxially along the artery itself, continued from the inguinal ligament distally, and aimed at the medial aspect of the knee. The incision can be extended superiorly or inferiorly to increase arterial exposure as necessary to achieve optimal inflow.

Alternatively, especially in obese patients with substantial abdominal pannus, a curvilinear incision can be placed 1 cm below and parallel to the inguinal ligament to avoid potential skin maceration and wound complications that may accompany vertical incisions in this situation. Although the proximal superficial femoral and deep femoral arteries can be exposed via this incision, such a curvilinear or oblique incision limits further distal arterial exposure. It therefore would not be selected if an extensive common and deep femoral artery endarterectomy is anticipated as potentially necessary to optimize inflow. The incision is carried sharply through the subcutaneous tissue and superficial fascia.

Investigating the state of the art for dissection and control of the common, superficial, and proximal deep femoral arteries, deep to the subcutaneous tissue and superficial fascia, the dissection is extended longitudinally, even when using an oblique incision, to optimize the length of femoral exposure. Depending on the depth of dissection and subcutaneous adiposity, self-retaining Weitlaner or cerebellar retractors are carefully placed to optimize exposure while avoiding traction injury to femoral nerve branches or the common femoral vein. Further dissection through the femoral sheath exposes the anterior surface of the femoral artery.

The dissection plane should remain centered directly over the femoral artery. Encountering venous structures indicates medial deviation from the optimal plane; exposure of the iliopsoas muscle, femoral nerve fibers, or lymphatic vessels is an indication of lateral deviation. An increasing incidence of femoral incisional complications, including wound edge necrosis and separation, lymphatic leaks, femoral neuropraxia, and venous injuries are associated with incorrectly placed inguinal incisions for femoral exposure.

In one method, dissecting directly along the CFA both proximally and distally and placement of silastic vessel loops around the femoral artery and its larger branches aids in retraction, dissection, and mobilization. Proximal dissection is continued along the CFA to the inguinal ligament. The inguinal ligament may be divided to aid in exposure or to enable extended endarterectomy. Caution is necessary in this area, as a prominent femoral vein tributary crosses anteriorly over the CFA in this area and is prone to injury if not identified, ligated, and divided early in the dissection. Inadvertent injury to this “vein of pain” produces retraction and troublesome bleeding. The medial and lateral femoral circumflex arteries, important collaterals in iliofemoral arterial occlusive disease, are identified at level of the inguinal ligament and individually controlled with removable clips or silastic vessel loops. Use of the former reduces clutter in the wound during endarterectomy or creation of the proximal anastomosis.

As the dissection proceeds distally, an abrupt change in caliber marks the femoral bifurcation and the origins of the deep and superficial femoral arteries (SFA). The latter continues distally in the same plane; the former usually courses posteriorly and laterally away from the femoral bifurcation. After silastic loops are placed on each vessel, gentle upward traction on the CFA or SFA may help bring the deep femoral artery into view. The lateral circumflex iliac vein may course anteriorly over the origin of the deep femoral artery and should be ligated and divided to optimize exposure and control of the first segment of this vessel.

Medial and distal dissection provides extended exposure of the proximal SFA. This vessel only occasionally has small branches in its proximal segment. A sensory branch of the femoral nerve may be present crossing the SFA from lateral to medial. Transection may result in medial thigh discomfort. Even extended femoral bifurcation dissections rarely require division of femoral nerve branches, which should be avoided to minimize postoperative paresthesias and dysesthesias.

Exposure of the distal portions of the deep femoral artery often enables use of shorter vein conduit in distal leg bypass or may improve outflow from proximal revascularization procedures (iliac angioplasty and stenting or aortofemoral bypass). These segments are easily exposed from either posteromedial or anteromedial approaches. The approach should be dictated by the indication (inflow sources or outflow target); an additional consideration is the necessity to obtain exposure in a native field, either in the setting of prior dissection or femoral graft infection.

Incisions are placed along either the medial (anteromedial approach) or lateral borders (posterolateral approach) of the sartorius muscle. The dissection plane is developed through the subcutaneous tissue and fascia, passing lateral or medial to the sartorius, respectively. Mobilize and retract sartorius muscle laterally or medially, depending on approach.

When the artery in the groin, the femoral artery, is stenosed or blocked, an operation can be used to remove the plaque from the artery in order to improve flow and circulation. The artery is close to the surface here under the skin, and therefore the surgical procedure is not too invasive. An alternative treatment would be angioplasty with a stent if required. This can be done with access to the arterial system from the opposite groin or arm to perform this.

Traditionally the long term results of surgery to remove the plaque when it is localized to the femoral artery have been better than angioplasty. However, in certain patient's not suitable for surgery, and with improving technology, angioplasty is being used more frequently.

The surgical procedure can be performed under general or local anesthetic. The plaque is removed from the artery. A patch is often used to repair the artery and avoid narrowing. Normally, patients will stay in hospital 1 or 2 nights following this procedure. If there is also disease with narrowed or blocked arteries elsewhere above the groin or further down the leg, the endarterectomy operation (removal of material on the inside of the artery) can be combined with angioplasty and stenting to treat these other areas at the same time. This combined or “hybrid” procedure is more common now and it achieves a better restoration of the circulation than the endarterectomy procedure alone.

Thus, in the current state of lower extremity revascularization for patients with claudication and critical limb ischemia, one method is treatment of the superficial femoral artery. Regarding treatment of the superficial femoral artery (SFA) for claudicative diseases, currently revascularization through an endovascular approach has become the default method for therapy in most, if not all, vascular beds (intricate network of minute blood vessels that ramifies through the tissues of the body or of one of its parts). The treatment for peripheral arterial obstructive disease in the lower extremity is no exception.

The quandary faced by the industry, and thus many of the questions asked revolve around whether or not to treat only when the claudication is at its worse or treat more aggressively and early, as this impacts potential heart-healthy lifestyles. Other key issues broach whether drug-delivery balloons and the current stent designs are not the correct systems for the SFA and whether changes to stent technologies—woven stents, alternatively designed stents or very conformable stents—define the future treatment directives.

Peripheral arterial disease (PAD) of the superficial femoral artery (SFA) is the most common cause of intermittent claudication. Atherosclerotic disease of the SFA is localized to the region of Hunter's canal. An isolated occlusion or stenosis of the SFA often results in decreased perfusion of the leg, resulting in demand related, reversible, ischemic pain localized to the calf. Ischemic rest pain and tissue loss, also known as critical limb ischemia (CLI), are uncommon manifestations of isolated SFA disease. CLI is more commonly observed when occlusive disease of the SFA is combined with occlusive disease involving the below knee popliteal artery or tibial arteries.

As discussed above, percutaneous transluminal angioplasty (PTA) is a minimally invasive technique for treatment of superficial femoropopliteal artery (SFA) obstructions or occlusions in patients with intermittent claudication as well as critical limb ischemia. With the introduction of endovascular stents, the problems of elastic recoil and residual stenoses due to arterial dissection could be resolved and initial reports of stenting for the treatment of occlusive atherosclerotic disease of the SFA have exhibit positive results. However, subsequent studies demonstrated that exaggerated neo-intimal hyperplasia (proliferation and migration of vascular smooth muscle cells primarily in the tunica intima, resulting in the thickening of arterial walls and decreased arterial lumen space) in the stented segment frequently leads to instent restenosis.

In one embodiment, the limb salvage technique may comprise the steps of proceeding antegrade or retrograde access through the post tibial artery, anterior tibial artery or pedal arch, utilizing a set of 0.014 floppy wires, in one embodiment hydrophilic guide wires may be utilized; advancing antegrade wire through the plantar arch and retrograde into the parallel artery; utilizing crossing catheters; utilizing atherectomy devices; utilizing angioplasty —long balloons; utilizing low pressure inflations; utilizing long inflation times and utilizing a stenting mechanism.

The access point to treat SFA is thus located behind the knee at the popliteal artery, which branches off from the femoral artery and is located in the knee and the back of the leg. Its courses near the adductor canal and the adductor hiatus, distinctive open areas inside the thigh.

An abrupt change in caliber marks the femoral bifurcation and the origins of the deep and superficial femoral arteries (SFA). The latter continues distally in the same plane; the former usually courses posteriorly and laterally away from the femoral bifurcation. After silastic loops are placed on each vessel, gentle upward traction on the CFA or SFA may help bring the deep femoral artery into view. The lateral circumflex iliac vein may course anteriorly over the origin of the deep femoral artery and should be ligated and divided to optimize exposure and control of the first segment of this vessel.

The popliteal artery is the direct continuation of the superficial femoral artery in the popliteal fossa as the vessel courses posteriorly behind the knee. It gives a main branch as anterior tibial artery and continue as tibioperoneal or tibiofibular trunk or posterior tibial artery. It supplies knee, lower leg and foot. The popliteal artery is a deeply placed continuation of the femoral artery after it passes through the adductor hiatus, or opening in the distal portion of the adductor magnus muscle. It courses through behind the knee the popliteal fossa and ends at the lower border of the popliteus muscle, where it branches into the anterior and posterior tibial arteries.

The deepest (most anterior) structure in the fossa, the popliteal artery runs in close proximity to the joint capsule of the knee as it spans the intercondylar fossa. Five genicular branches of the popliteal artery supply the capsule and ligaments of the knee joint. The genicular arteries are the superior lateral, superior medial, middle, inferior lateral, and inferior medial genicular arteries. They participate in the formation of the periarticular genicular anastomosis, a network of vessels surrounding the knee that provides collateral circulation capable of maintaining blood supply to the leg during full knee flexion, which may kink the popliteal artery.

Because the popliteal artery is deep, it may be difficult to feel the popliteal pulse. Palpation of this pulse is commonly performed with the person in the prone position with the knee flexed to relax the popliteal fascia and hamstrings. The pulsations are best felt in the inferior part of the fossa where the popliteal artery is related to the tibia. Weakening or loss of the popliteal pulse is a sign of a femoral artery obstruction.

A popliteal aneurysm (abnormal dilation of all or part of the popliteal artery) usually causes edema and pain in the popliteal fossa. A popliteal aneurysm may be distinguished from other masses by palpable pulsations (thrills) and abnormal arterial sounds (bruits) detectable with a stethoscope. Because the artery lies deep to the tibial nerve, an aneurysm may stretch the nerve or compress its blood supply (see vasa vasorum). Pain from such nerve compression is usually referred, in this case to the skin overlying the medial aspect of the calf, ankle or foot. Because the artery is closely applied to the popliteal surface of the femur and the joint capsule, fractures of the distal femur or dislocations of the knee may rupture the artery, resulting in hemorrhage. Furthermore, because of their proximity and confinement within the fossa, an injury of the artery and vein may result in an arteriovenous fistula (communication between an artery and a vein). Failure to recognize these occurrences and to act promptly may result in the loss of the leg and foot. If the femoral artery must be ligated, blood can bypass the occlusion through the genicular anastomosis and reach the popliteal artery distal to the ligation.

Further, popliteal artery entrapment syndrome is a rather uncommon pathology, which results in claudication and chronic leg ischemia. The popliteal artery may be compressed behind the knee, due to congenital deformity of the muscles or tendon insertions of the popliteal fossa. This repetitive trauma may result in stenotic artery degeneration, complete artery occlusion or even formation of an aneurysm.

Advancing distal to proximal can reduce shower of emboli or particle flow or other such events causing pieces of material to create a further Blockage inside a blood vessel. The embolus may be a blood clot (thrombus), a fat globule (fat embolism), a bubble of air or other gas (gas embolism), or foreign material below the knee into the territory of the foot. Positioning an embolic protection device at or near the puncture site will permit safe removal of dislodged plaque or emboli generated during the access and angioplasty procedure.

Thrombectomy and embolectomy are life-saving procedures mostly performed in emergency situations. The terms embolectomy and thrombectomy are sometimes used interchangeably, but there are some differences between the two. To understand how a thrombectomy or embolectomy is performed, you must first understand why they are done.

Sometimes, due to various factors like disease, blood clots can form in the blood vessels. A thrombus is usually a solid-mass stationary clot. An embolus is when part, or all, of that clot is dislodged and begins to travel through the circulatory system. Essentially, an embolus is a moving thrombus. These clots can pose serious and even fatal risks.

When an artery is obstructed by a thrombus or embolus, it is called a thromboembolism or embolism. Types of embolisms include the following:
Thromboembolism—A formation in a blood vessel by a blood clot that has become dislodged from another site and carried through the bloodstream
Cholesterol embolism—Blockage of a blood vessel as the result of atherosclerotic plaque
Fat embolism—Blockage of a blood vessel caused by fat or bone fractures
Air embolism—Obstruction of a blood vessel by gaseous matter, such as an air bubble
Septic embolism—A bacteria-containing pus blockage of a blood vessel.
Tissue embolism—A blockage of a blood vessel formed by natural tissues within the body
Foreign body embolism—A blockage of a blood vessel that wasn't naturally produced by the body
Amniotic fluid embolism—An obstruction of a blood vessel formed by amniotic fluid, fetal cells, hair or other debris that have entered the mother's bloodstream

A thrombectomy is the removal of a thrombus and an embolectomy is the removal of an embolus and there are two main types of embolectomy and thrombectomy, depending on the blood vessel that needs treatment and the severity of the condition as follows:

### Catheter-Based Procedures:

Catheter-based procedures involve passing a small tube through a tiny incision into the clot site and using special instruments to remove the clot by using balloon embolectomy or aspiration embolectomy. A balloon embolectomy may be accomplished by inserting a catheter with a small inflatable balloon attached at the end into the vein and past the clot. The balloon is then inflated and slowly pulled back out of the vein, to remove the clot simultaneously. Additionally, an aspiration embolectomy is performed by using suction to remove the thrombus from the vein.

In an additional procedure, Open surgery involves making a larger incision in the area of the blood clot through the blood vessel to remove it. Open surgery is less common but is sometimes the best choice for emergencies to save an organ or in other cases.

Weighing the risks and complications of not treating, the obstructions within the vein go untreated, some serious complications can occur. These may include a pulmonary embolism occurs when a blood clot travels up to a vein within a lung from another part of the body, causing a blockage. It is crucial to be aware of the warning signs of a pulmonary embolism, as it can be fatal. Symptoms or signs of a pulmonary embolism include a chest pain or discomfort that worsens when you take a deep breath or when you cough, unexplained sudden onset of shortness of breath, rapid pulse, feeling lightheaded or dizzy, fainting and coughing up blood.

Another risks and complications of not treating could be postphlebitic syndrome which occurs as a complication of damage to the vein caused by a blood clot. The damage results in inhibited blood flow in the affected areas of the vein. Signs and symptoms include leg pain, Swelling of the legs, Skin sores, Skin discoloration

Also, cutting balloons may be suited for as the balloon-mounted microtomes guarantee smooth lumen gain within the stent, without the risk of vessel wall perforation. Initial reports of the use of the cutting balloon for the treatment of occlusive atherosclerotic disease of the SFA show promising results, indicating that the problems of elastic recoil and residual stenoses due to arterial dissection might be resolved. The cutting balloon has four tiny microtomes (<0.1 mm height) on the outside, which cut the fibrous plaque during expansion of the balloon. Consequently, the problem of elastic recoil is ideally addressed, additionally less trauma is exercised on the vessel wall during dilatation of the balloon.

This might be achieved by a reduction of vessel wall trauma, vessel wall inflammation and consequently reduced neointimal formation. Although the indications for CB-PTA in the SFA includes significant residual stenosis or in-stent restenosis, there are currently no published randomized controlled trials (RCT) comparing PTA vs. cutting balloon angioplasty (CB-PTA) for any specific condition. This lack of data led us to initiate a RCT comparing primary PTA vs. CB-PTA for treatment of in-stent restenosis in patients with intermittent claudication or critical limb ischemia with TASC category A-B in the femoropopliteal artery.

Historically, endovascular treatment of the SFA was first described by Charles Dotter wherein he used Teflon coated dilators to sequentially angioplasty the SFA in an 82-year-old woman to treat critical limb ischemia that was considered non-operable. Subsequently, Gruntzig popularized the concept of catheter directed balloon angioplasty. Angioplasty disrupts the atherosclerotic plaque by displacing it radially and results in stretching of the adventitia thereby increasing the lumen diameter in the treated vessel. By definition, a dissection is created and if significant, can be flow limiting.

Currently, the most commonly utilized endovascular revascularization options are percutaneous transluminal angioplasty (PTA) with provisional stenting or primary stenting. Provisional or selective stenting is indicated for the treatment of flow limiting dissections and/or persistent, hemodynamically significant stenoses or recoil after PTA. This approach is recommended by the Tran-Atlantic inter-Society Consensus document II (TASC II) when treating SFA disease. However, both PTA alone and primary stenting can successfully treat SFA disease. Therefore, the debate continues as to which endovascular treatment is superior.

PTA has the advantage of being inexpensive and technically simpler than primary stenting, and is especially well looked upon as only PTA alone avoids utilization of foreign bodies that may be a potential stimulus for intimal hyperplasia. Another benefit of using PTA alone includes avoiding material fatigue and fractures associated with stenting wherein fatigue and fracture may result from the torque and deformation of the femoropopliteal arteries that occurs during flexion of the knee joint.

Further, the advent of lower profile angioplasty balloons allows PTA of the SFA through vascular sheaths as small as 4 French. Smaller sheath diameters result in fewer complications, and therefore, are considered safer. In addition, the ease of re-intervention or bypass of an angioplastied arterial segment following PTA may be advantageous. The presence of a stent may impede endovascular re-intervention if a re-stenosis results in occlusion of the stented arterial segment. Moreover, angioplasty preserves collateral vessels that may be compromised by stent placement.

The clear need for advancement is illustrated through the need to treat chronic limb ischemia arise when it results in the limb threatening conditions of rest pain and tissue loss and possible loss of limb illustrate the exigency. Debilitating symptoms of intermittent claudication, a condition in which cramping pain in the leg is induced by exercise, typically caused by obstruction of the arteries, may occur and intermittent claudication is the most common manifestation of isolated SFA arterial disease. Though SFA disease is often present in patients with CLI, it is frequently seen in conjunction with multi-level arterial occlusive disease.

Remote superficial femoral artery endarterectomy (RSFAE) is a procedure to remove plaques inside the superficial femoral artery (SFA). The femoral artery is the main blood vessel in your thigh that carries blood and oxygen to the legs. Plaques are fat, cholesterol, or tissues that are clogged in the inner wall of the artery. When plaques build up inside the superficial femoral artery, blood flow to the legs may be decreased. RSFAE may be done to relieve problems caused by a narrowed or blocked artery. Problems that may happen include severe pain in the hip, thigh, calf, or foot, and trouble when walking. Having these problems may decrease a person's ability to do his daily activities and affect his quality of life.

With RSFAE, the plaque that blocks the artery is removed through a small incision (cut) in the groin. The groin is the area where your abdomen (stomach) meets your upper leg. Caregivers strip, cut, and remove the plaque by using different tools inserted through the SFA. This may be accomplished by using a special type of x-ray as a guide. RSFAE may be followed by other procedures, such as angioplasty and stenting to open the artery using a small, high pressure balloon and implant a metal or plastic stent, in the area where the blockage was removed, to keep the artery open.

In an advantageous manner, in utilizing a retrograde approach to treat the SFA with a PTA approach augments the overall capabilities. As an alternative, a guide catheter can be advanced in stages with a balloon or a guidewire can be advanced independently to access the most proximal location in the vessel prior to introduction of the balloon catheter and paving can advance proximal to distal, in the direction of the puncture site.

### SUMMARY OF THE INVENTION

The instant apparatus and system, as illustrated herein, is clearly not anticipated, rendered obvious, or even present in any of the prior art mechanisms, either alone or in any combination thereof. A versatile and low-profile system, method and series of apparatuses for creating and utilizing a system which allow at or below the ankle stenting for a non-invasive treatment of critical limb ischemia. Thus, the several embodiments of the instant apparatus are illustrated herein.

The present invention provides a system, methods and accompanying apparatuses for the treatment of limb and life-threatening disease scenarios including peripheral arterial disease (PAD) and the most severe manifestation of PAD, namely Critical Limb Ischemia (CLI) which may be resulting in ulcers, gangrene, amputation and death. With such a low-profile system, CLI patients will eventually be able to receive treatment in an office-based outpatient setting which allows for minimal bleeding.

It is therefore an objective of the instant system to introduce a novel system or platform for treating PAD and CLI utilizing antegrade and retrograde advancement.

It is therefore an objective of the instant system to introduce a 3 mm by 60 mm device stent may be inserted into Left Popliteal and a Self-expanding sheath may be deployed in the distal posterior tibial artery from the lateral plantar.

It is therefore an objective of the instant system to introduce a 3 mm by 60 mm device stent.

It is therefore an objective of the instant system to introduce a 3 mm by 60 mm device stent which may be inserted into Left Popliteal.

It is therefore an objective of the instant system to introduce a 3 mm by 60 mm device stent which may be inserted into Left Popliteal and utilizing a self-expanding sheath, which may be deployed in the distal posterior tibial artery.

It is therefore an objective of the instant system to introduce a stent which may sustain high filling pressure to the microcirculation for better and faster wound healing.

It is therefore, an objective of the instant system to introduce a stent which may conform to different arterial diameters from 1.5 mm to 4 mm.

In one embodiment, it is therefore an objective of the instant system to introduce a 4 Fr. compatible stent used in conjunction with a one hundred- and twenty-centimeter (“120 cm”) length delivery catheter, as well as accompanying stent devices disposed in numerous lengths to meet individual patient needs.

In one embodiment, it is therefore an objective of the instant system to introduce a 4 Fr. compatible stent used in conjunction with a one hundred- and twenty-centimeter (“120 cm”) length delivery catheter, as well as accompanying stent devices disposed in numerous diameters to meet individual patient needs.

In one embodiment, it is therefore an objective of the instant system to introduce a 4 Fr. compatible stent used in conjunction with a one hundred- and twenty-centimeter (“120 cm”) length delivery catheter, as well as accompanying stent devices disposed in up to five lengths to meet individual patient needs.

In one embodiment, it is therefore an objective of the instant system to introduce a 4 Fr. compatible stent used in conjunction with a one hundred- and twenty-centimeter (“120 cm”) length delivery catheter, as well as accompanying stent devices disposed in up to diameters to meet individual patient needs.

### BRIEF DESCRIPTION OF THE DRAWINGS

The invention may be more completely understood in consideration of the following detailed description of various embodiments of the invention in connection with the accompanying drawings, in which:

FIG. 1 illustrates a front perspective cross sectional view of a lower limb and further illustrates a clogged structure where in critical limb ischemia has set in and wherein the second toe is corroded.

FIG. 2 further illustrates a big toe with sores which come from critical limb ischemia and at this point the patient may be looking at an amputation in prior years.

FIG. 3A illustrates a side cross sectional view of the entire vascular anatomy of the human foot in order illustrate the artery structure and this further to illustrate the path for instant invention will be launched to wrap around the arch.

FIG. 3B illustrates a top perspective cross sectional view of the entire vascular anatomy of the human foot in order illustrate the artery structure and this further to illustrate the path for instant invention will be launched to wrap around the arch.

FIG. 4A illustrates a front view of the entire vascular anatomy of the human foot in order illustrate the artery structure and this further to illustrate the path for instant invention will be launched to wrap around the arch.

FIG. 4B illustrates a diabetic foot ulcer figuration illustrating the clogged and outwardly extending arteries.

FIG. 5 illustrates the guide wire, single lumen and other components.

FIG. 6 illustrates one embodiment of a feasible component to be utilized in a low profile atherectomy procedure, a CSI Diamondback MicroCrown 1.25 mm.

FIG. 7 illustrates one embodiment of a feasible namely a Spectranetics TurboElite 0.9 mm.

FIG. 8A-8E illustrate a Pedal Arch Revascularization.

FIG. 9A-9D Progression of placing a stent across the ankle joint.

FIG. 10A-10C illustrate the progressive release of the stent across the ankle joint and the simultaneous backing off the catheter and/or sheath, within the confines of the current system for access across the pedal arch.

FIG. 11A-11C illustrate issues concerning stenosis and occlusion of the proximal PTA, the distal PTA and the lateral planar.

FIG. 12A-12B illustrate catheters traversing the arch within the confines of the current system for access across the pedal arch.

FIG. 13A-13B illustrate use of a laser in the PT posterior tibial veins and the posterior tibial artery PTA within the confines of the current system for access across the pedal arch.

FIG. 14A-14B illustrate usage of a 2.0 mm Ampherion balloon within the confines of the current system for access across the pedal arch.

FIG. 15A-15D illustrate 2.0 mm Ampherion balloon of PTA and restoration of blood flow to the pedal arch area.

FIG. 16A-16B illustrates the proximal posterior tibial artery and the posterior tibial artery.

FIG. 17A-17D illustrate the new specialty self-expanding stent deployed from the later planar to the distal posterior tibial artery. FIG. 17A illustrates the initial phase of the release of the stent for new specialty self-expanding stent deployed from the later planar to the distal posterior tibial artery. FIG. 17B illustrates the second phase of the release of the stent for the new specialty self-expanding stent deployed from the later planar to the distal posterior tibial artery. FIG. 17C illustrate the third phase of the release of the stent for the new specialty self-expanding stent deployed from the later planar to the distal posterior tibial artery. FIG. 17D illustrates the final phase of the release of the stent for the new specialty self-expanding stent deployed from the later planar to the distal posterior tibial artery.

FIG. 18A-18B illustrate the tibio-pedal stent deployed from the later planar to the distal posterior tibial artery. FIG. 18A illustrates the initial phase of the release of the tibio-pedal stent for one embodiment of the new specialty self-expanding stent deployed from the later planar to the distal posterior tibial artery. FIG. 18B illustrates the second phase of the release of the stent for one embodiment of the for the new specialty self-expanding stent deployed from the later planar to the distal posterior tibial artery.

FIG. 19A-19B illustrate how one embodiment of the instant novel stent is deigned to sustain high filling pressure to the microcirculation for better and faster wound healing. Further illustrated is the continued path of the tibio-pedal stent deployed from the later planar to the distal posterior tibial artery. FIG. 19A illustrates the continued phase of the release of the tibio-pedal stent for one embodiment of the new specialty self-expanding stent deployed from the later planar to the distal posterior tibial artery. FIG. 19B illustrates the final fully extended phase of the release of the stent for one embodiment of the for the new specialty self-expanding stent deployed from the later planar to the distal posterior tibial artery.

FIG. 20A-20B illustrate one embodiment of the stent mechanism and illustrate the flexibility of the stent mechanism within the system. FIG. 20A illustrates how the stent conforms to the different arterial diameters, which met with the inherent design character required in the design process, in order to accommodate going from 1.5 mm to 4 mm if required. FIG. 20B illustrates a scenario wherein the stent will dilate to the underline lumen or to the post dilated balloon. Additionally, illustrated is the stent transitioning from the planar artery to posterior tibial artery PTA

FIG. 21A-21C illustrate one embodiment of the stent mechanism in place and varying positions of Posterior Tibial Artery (PTA) balloons. FIG. 21A illustrates one position of a Posterior Tibial Artery (PTA) balloon. FIG. 21B illustrates a cross-section of an artery of the stent in place. Moreover, FIG. 21C illustrates one position of a Posterior Tibial Artery (PTA) balloon.

FIG. 22A-22B illustrate one embodiment of the stent mechanism in place and varying positions of Posterior Tibial Artery (PTA) balloons. FIG. 22A illustrates one position of a Posterior Tibial Artery (PTA) balloon. FIG. 22B illustrates a cross-section of an artery of the stent in place. Additionally, illustrated is the stent transitioning from the planar artery to posterior tibial artery PTA.

FIG. 23 illustrates the partially deployed stent illustrating the immediate wall opposition.

FIG. 24 illustrates a flow diagram for one embodiment of a method to treat critical limb ischemia.

FIG. 25 illustrates a flow diagram of another embodiment to treat critical limb ischemia.

### DETAILED DESCRIPTION OF THE SEVERAL EMBODIMENTS

The detailed description set forth below is intended as a description of presently preferred embodiments of the apparatus and does not represent the only forms in which the present apparatus may be construed and/or utilized. The description sets forth the functions and the sequence of the steps for producing the system and accompanying apparatus. However, it is to be understood that the same or equivalent functions and sequences may be accomplished by different embodiments also intended to be encompassed within the scope of the invention.

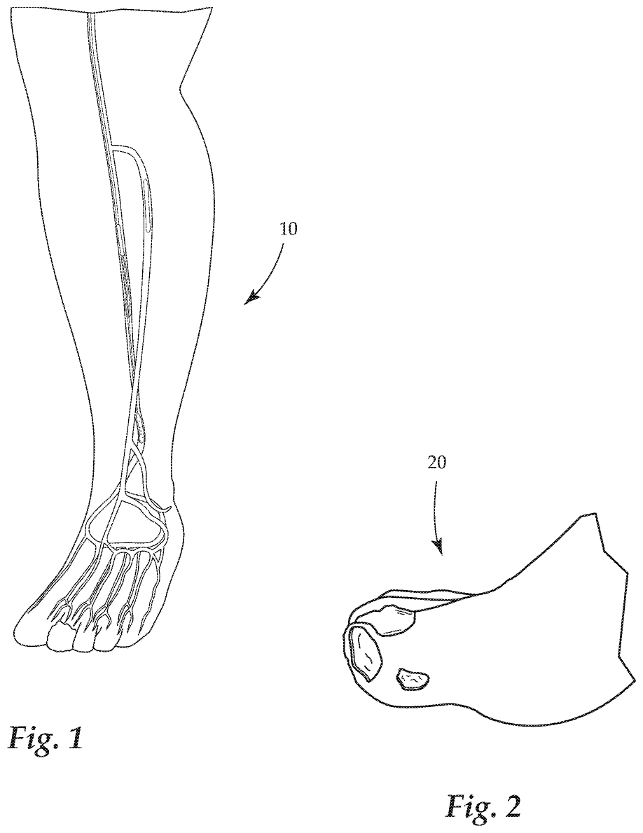

FIG. 1 illustrates a lower limb 10 and further illustrates a clogged structure wherein critical limb ischemia has set in and wherein the second toe is corroded. FIG. 2 further illustrates an example wherein a toe 20 with sores 89 which come from critical limb ischemia and at this point the patient may be looking at an amputation in prior years.

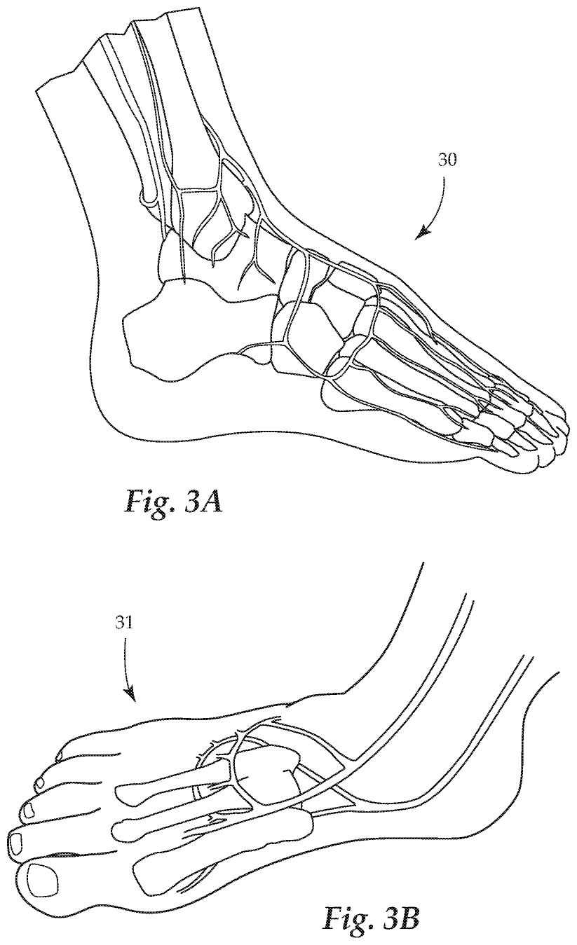

FIG. 3A shows the entire vascular anatomy of the human foot 30 in order illustrate the artery structure and this further to illustrate the path for instant invention will be launched to wrap around the arch to the posterior tibial artery, to the popliteal artery and then on to treat the clogging in the SFA. FIG. 3B utilizes top perspective cross sectional view of a more simplified area further illustrating the entire vascular anatomy of the human foot 31 in order illustrate the artery structure and the path for instant system to be introduced and wrap around the arch to the posterior tibial artery.

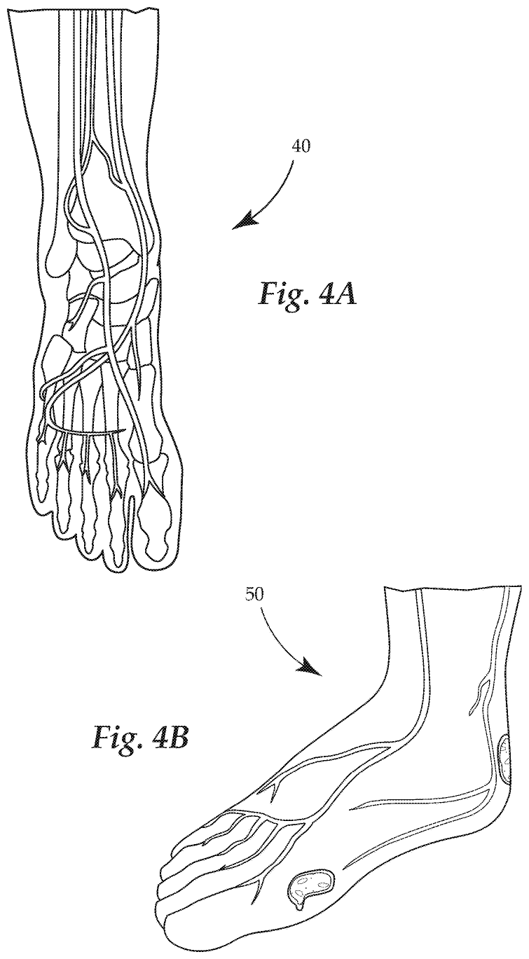

FIG. 4A shows the entire vascular anatomy of the human foot 40 in order illustrate the artery structure and this further to illustrate the path for instant invention will be launched to wrap around the arch. FIG. 4B illustrates a diabetic foot ulcer, further illustrating the clogged and outwardly extending arteries 50.

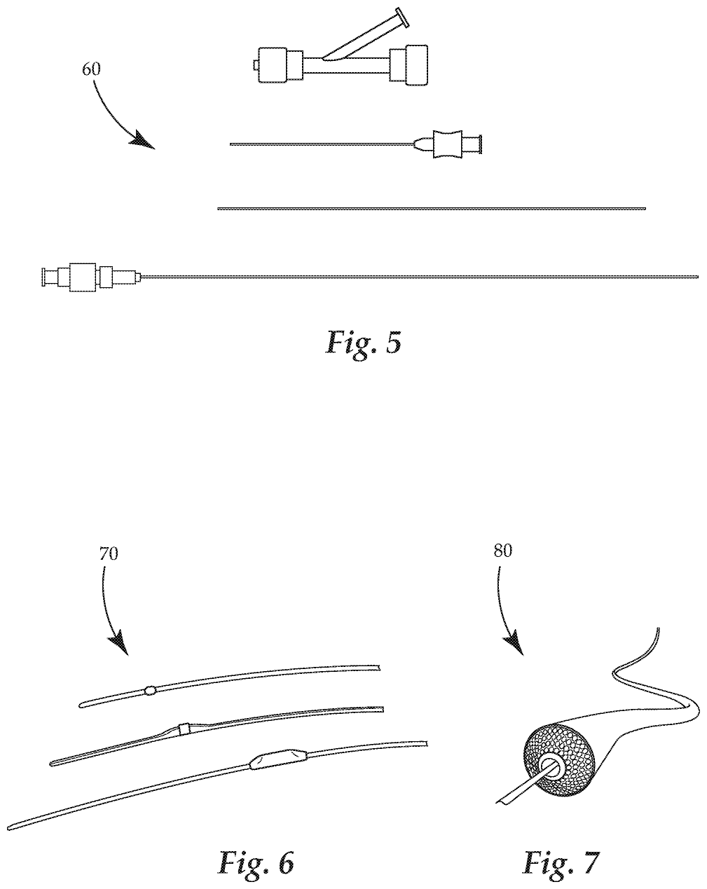

FIG. 5 illustrates the guide wire, single lumen and other applicable components for the delivery system 60. As illustrated in FIG. 5, gaining access may occur in several manners, and with differing tools, including but not limited to: antegrade access via PT or AT into ankle and foot and retrograde access via pedal or plantar arch into DP. In some embodiments, Micropuncture kits are very useful for pedal access and one example for use may be the Cook Micropuncture Access Kit.

In addition, crossing and support systems unitized may include wires and catheters, including 0.014 wires. In a preferred embodiment, the wires are hydrophilic wires selected from the group consisting of the Boston Scientific PT2 Support Guidewire, the Abbott Hi-Torque Command Guidewire, and the Approach Hydro ST MicrowireGuide. Compatible catheter systems may include the Spectranetics Quickcross Support Catheter and the Cook CXI Support Catheter.

FIG. 6 and FIG. 7 illustrate feasible components to be utilized in a low profile atherectomy procedure. FIG. 6 illustrates CSI Diamondback MicroCrown 1.25 mm 70. FIG. 7 illustrates a Spectranetics TurboElite 0.9 mm 80.

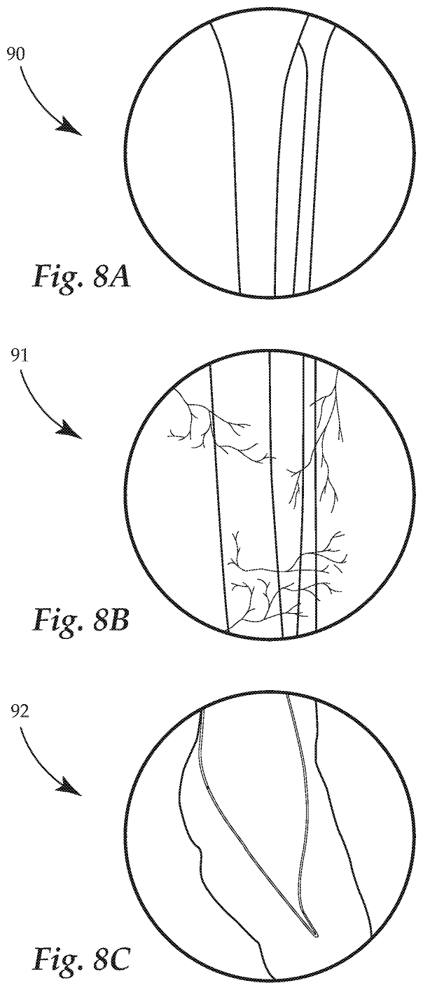

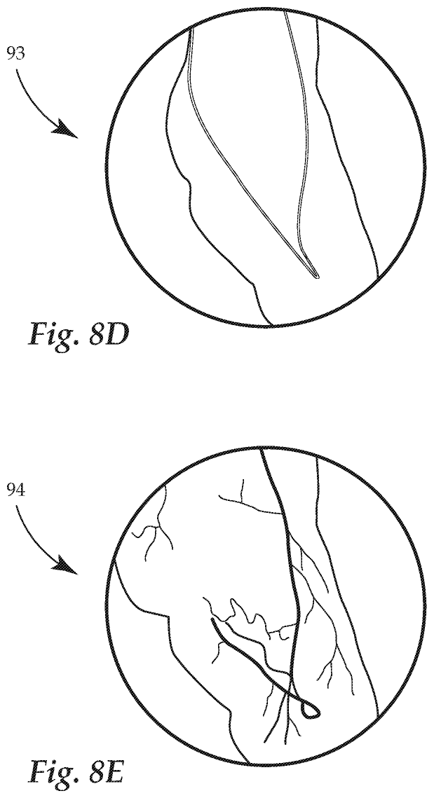

FIG. 8A-8E illustrate one example of a Pedal Arch Revascularization illustrating the steps from insertion to location and deployment of the stent mechanism. FIGS. 8A through 8E illustrate one version of a pedal arch revascularization process. FIG. 8A illustrates the area

In one embodiment, the system may be utilized for antegrade access via the Posterior Tibial Artery (PTA) or the Anterior Tibial Artery (ATA) into ankle and foot Angioplasty for the Pedal Arteries. One of the keys to utilization of the system is the use of Low Profile plain old balloon angioplasty (POBA), utilizing different configurations as follows: Medtronic Amphirion Deep with Tapered end, smallest size 1.5/2.0; Cordis Saber PTA, with the smallest size 2.0 mm; New DCB on the pipeline with Lutonix BTK DCB, using Smallest size 2.0. The above described construction is then utilized for complete revascularization.

FIG. BA illustrates the need for revascularization 90. Next, FIG. BE illustrates the partially revascularized area 91 illustrating some blood flow restored. FIG. BC illustrates the catheter and stent of the system 92 after displayed wrapped around the arch to the distal PTA and into the lateral planer.

FIG. BD further illustrates the system 93 reaching around the arch to the distal posterior tibial artery (PTA) and into the lateral planer as the area is revascularized. Lastly, FIG. BE illustrates the completely revascularized area 94 illustrating some blood flow restored.

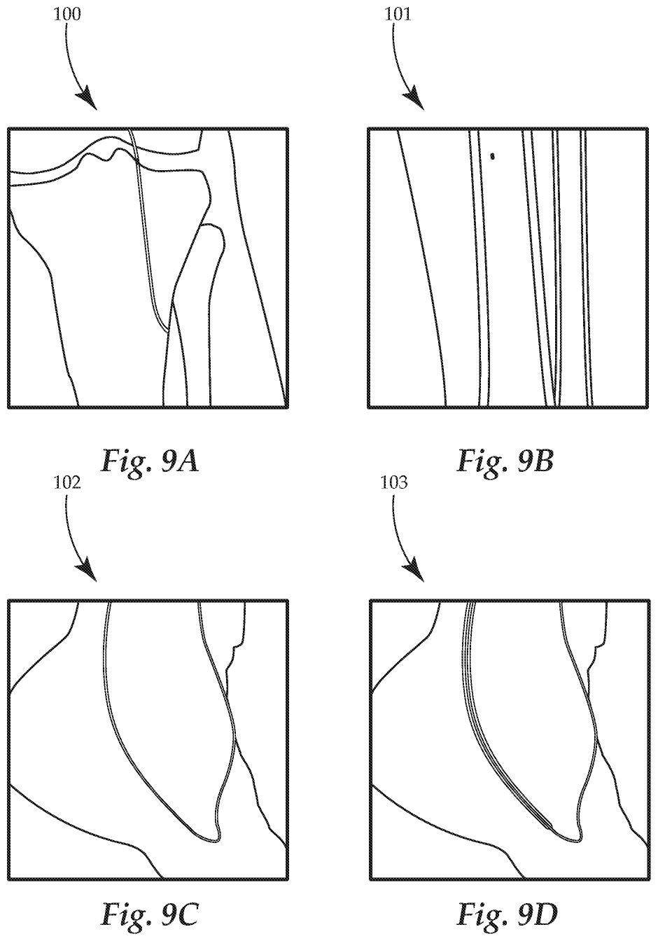

FIG. 9A-9D illustrate an additional iteration of a pedal arch revascularization, once again illustrating the steps from insertion 100 to location 101 and initial deployment 102 and final deployment 103 of the stent mechanism, within the confines of the current system.

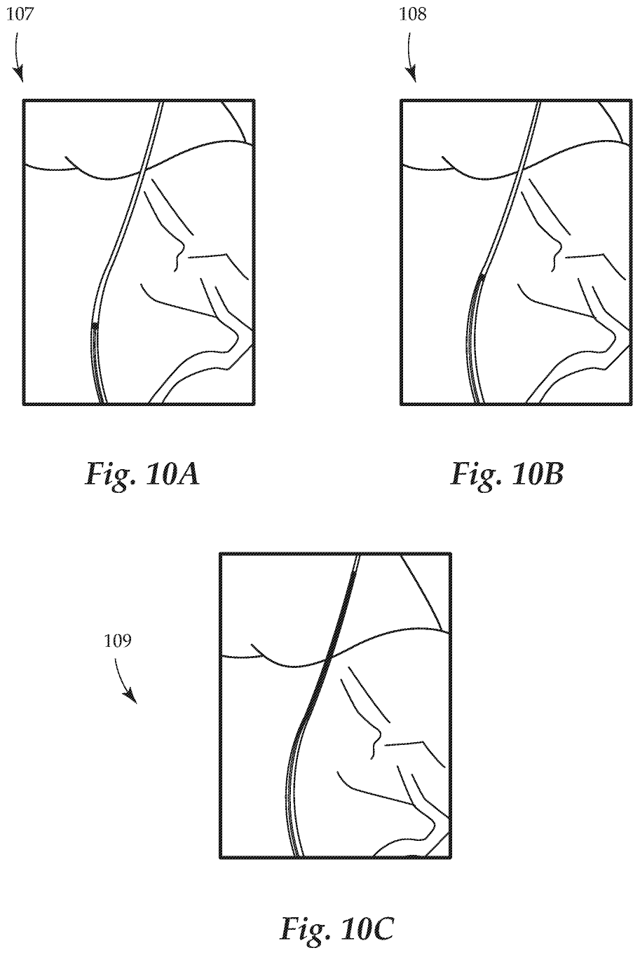

FIG. 10A-10C illustrate the progressive release of the stent across the ankle joint and the simultaneous backing off the catheter and/or sheath, within the confines of the current system for access across the pedal arch. FIG. 10A illustrates the initial stent releasing activity 107. FIG. 10B illustrates the continued release of the stent activity 108. FIG. 10C illustrates the completed release of the stent across the pedal arch 109.

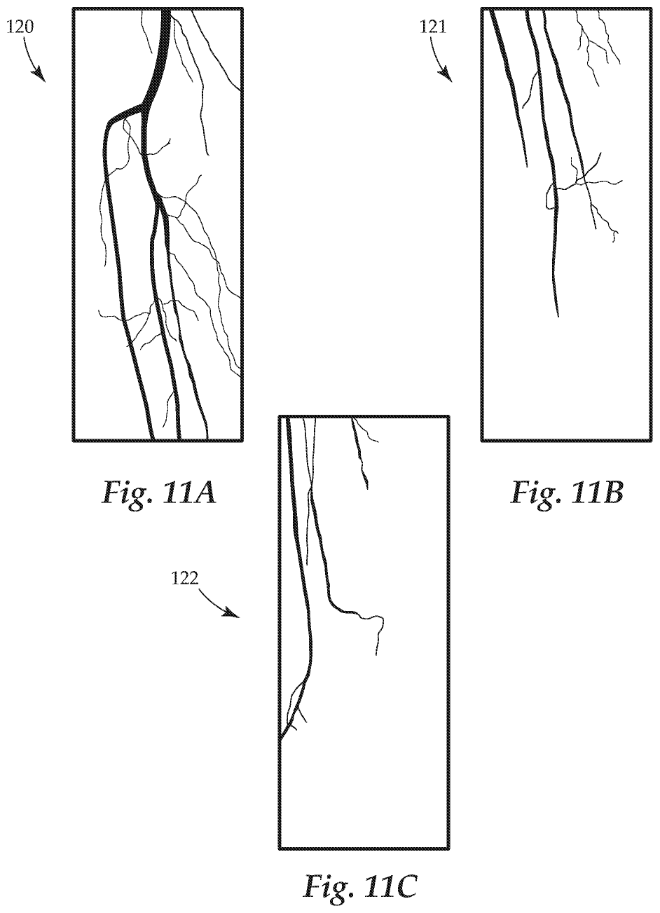

FIG. 11A-11C illustrate issues concerning stenosis and occlusion of the proximal PTA, the distal PTA and the lateral planar. FIG. 11A illustrates severe stenosis of the proximal PTA 120. FIG. 11B illustrates occlusion for the distal PTA 121. FIG. 11C illustrates totally occluded distal PTA into lateral planar 12.

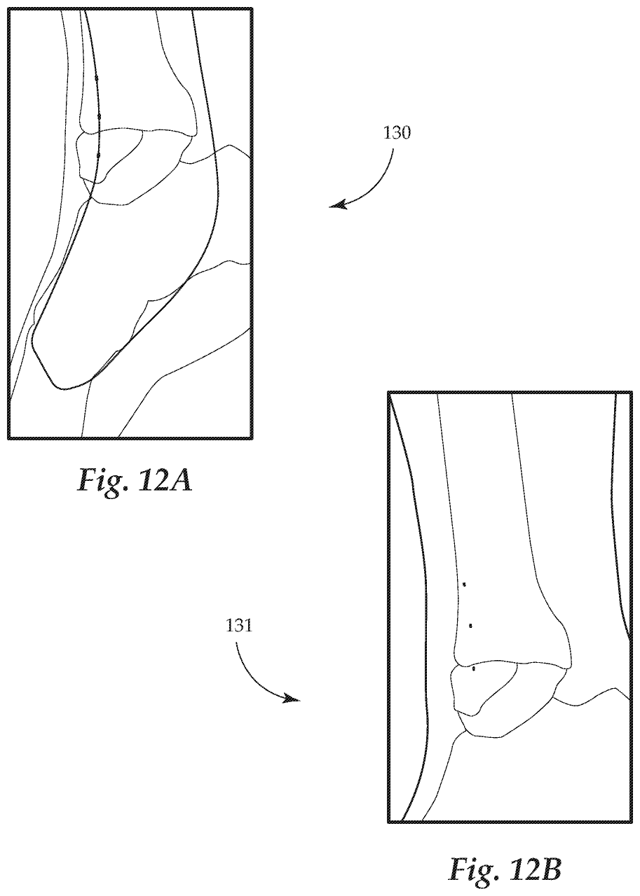

FIG. 12A-12B illustrate catheters traversing the arch within the confines of the current system for access across the pedal arch. FIG. 12A illustrates a catheter 130 traversing the lateral plantar branch, into pedal arch, through dorsalis pedis, back into the anterior tibial artery (ATA) in the anterior compartment of the leg. FIG. 12B illustrates a Quick-Cross® catheter 131 in distal ATA traversing the pedal arch.

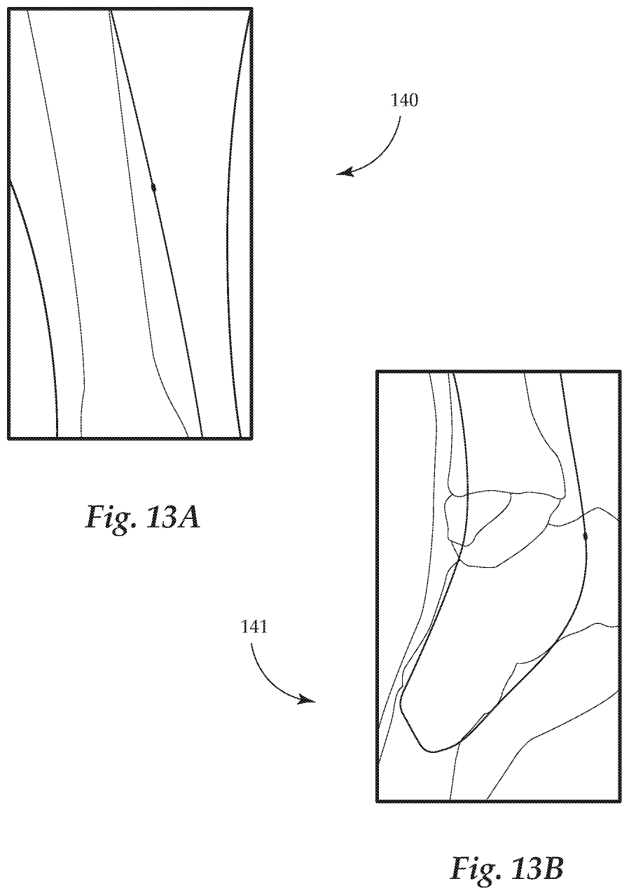

FIG. 13A-13B illustrate use of a laser in the PT posterior tibial veins and the posterior tibial artery PTA within the confines of the current system for access across the pedal arch. FIG. 13A illustrates use of a laser 140 in the posterior tibial veins (PT). FIG. 13B illustrate use of a laser 141 in the posterior tibial artery (PTA).

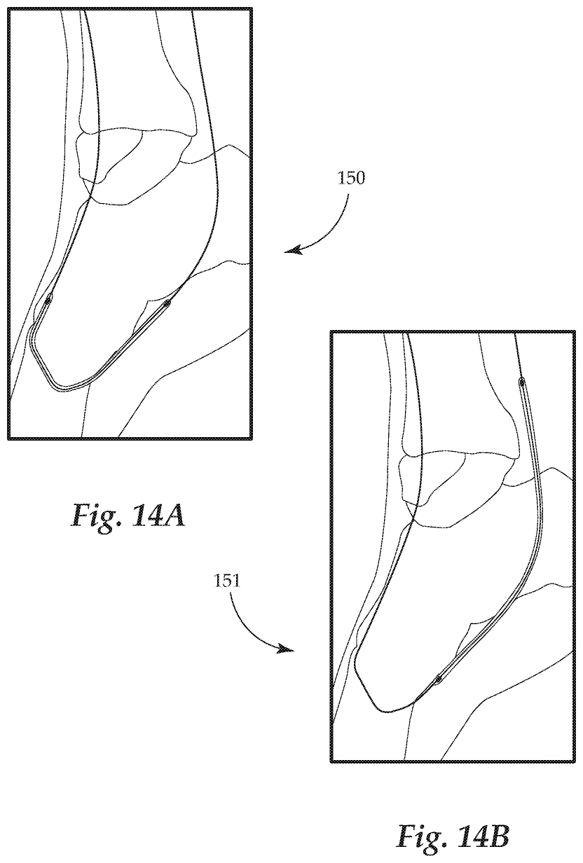

FIG. 14A-14B illustrate usage of a 2.0 mm Ampherion balloon within the confines of the current system for access across the pedal arch. FIG. 14A illustrates use of a 2.0 mm Ampherion balloon 150 at the lateral plantar artery and the pedal arch. FIG. 14B illustrates use of a 2.0 mm Ampherion balloon 151 of lateral plantar artery and distal PTA.

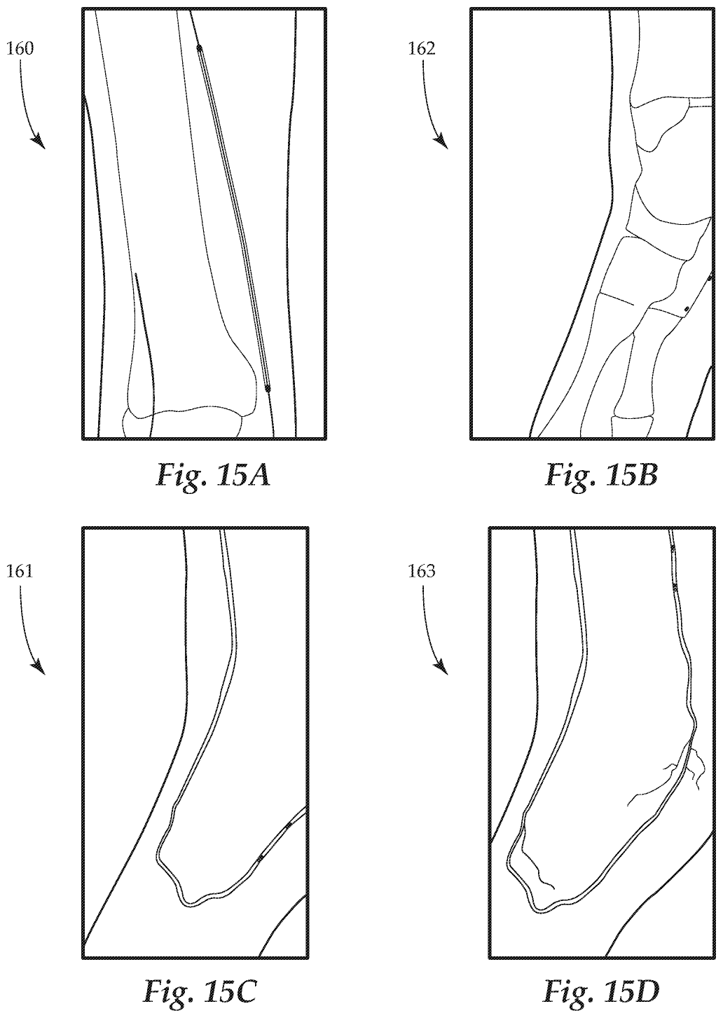

FIG. 15A-15D illustrate 2.0 mm Ampherion balloon of PTA and restoration of blood flow to the pedal arch area. FIG. 15A illustrates 2.0 mm Ampherion balloon 160 of the PTA. FIG. 15B illustrates blood flow restored to the pedal arch 161. FIG. 15C further illustrates blood flow restored to the pedal arch 162. FIG. 15D illustrates the Distal ATA, dorsalis pedis, pedal arch, lateral plantar and posterior tibial artery (PTA) 163.

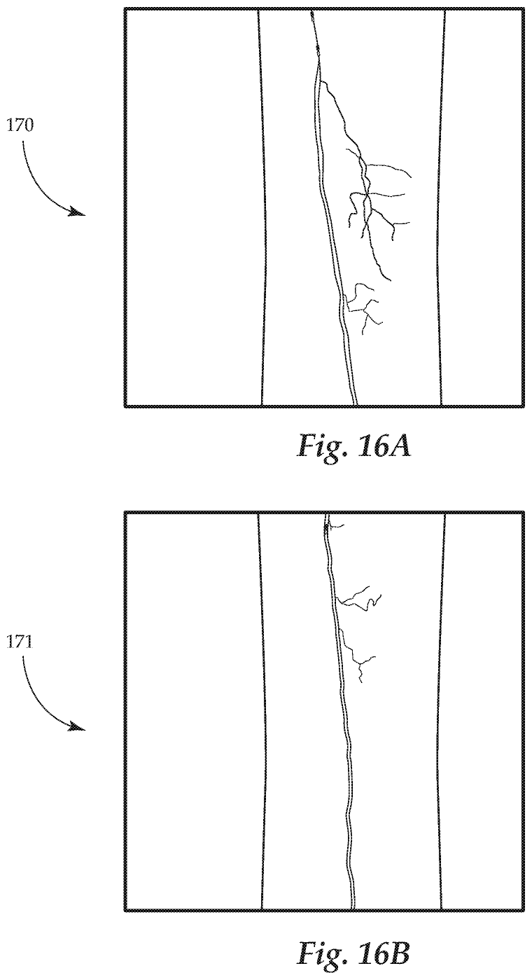

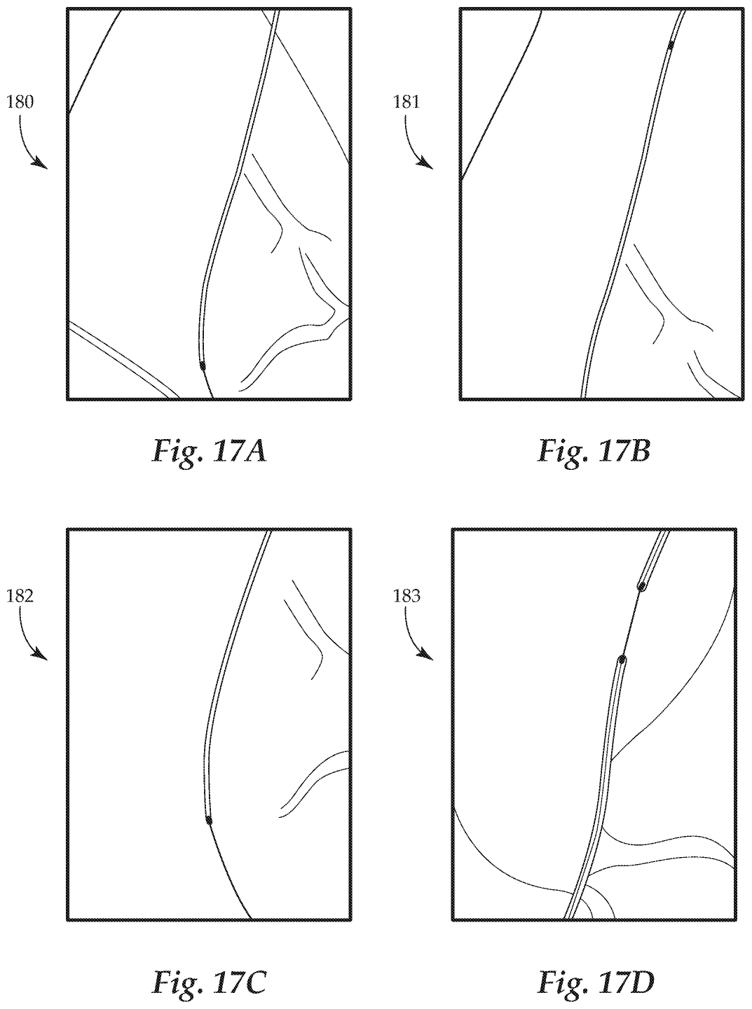

FIG. 16A illustrates the proximal posterior tibial artery 170. FIG. 16B illustrates the posterior tibial artery 171. FIG. 17A-D illustrate the new specialty self-expanding stent deployed from the later planar to the distal posterior tibial artery.

FIG. 17A illustrates the initial phase of the release of the stent for new specialty self-expanding stent 180 deployed from the later planar to the distal posterior tibial artery. FIG. 17B illustrates the second phase of the release of the stent 181 for the new specialty self-expanding stent deployed from the later planar to the distal posterior tibial artery. FIG. 17C illustrate the third phase of the release of the stent for the new specialty self-expanding stent 182 deployed from the later planar to the distal posterior tibial artery. FIG. 17D illustrates the final phase of the release of the stent 183 for the new specialty self-expanding stent deployed from the later planar to the distal posterior tibial artery.

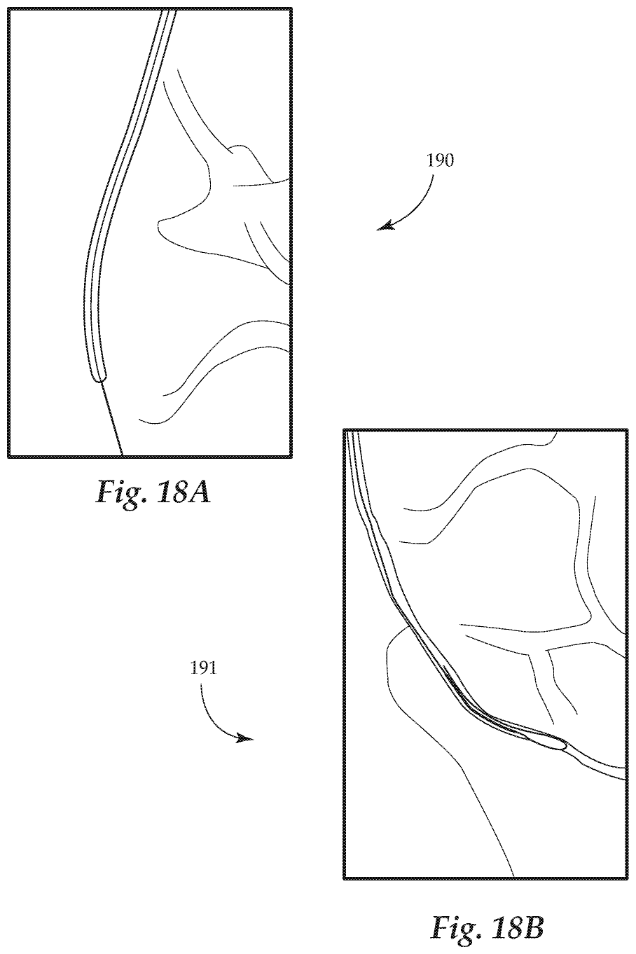

FIG. 18A-18B illustrate the tibio-pedal stent deployed from the later planar to the distal posterior tibial artery. FIG. 18A illustrates the initial phase of the release of the tibio-pedal stent 190 for one embodiment of the new specialty self-expanding stent deployed from the later planar to the distal posterior tibial artery. FIG. 18B illustrates the second phase of the release of the stent 191 for one embodiment of the for the new specialty self-expanding stent deployed from the later planar to the distal posterior tibial artery.

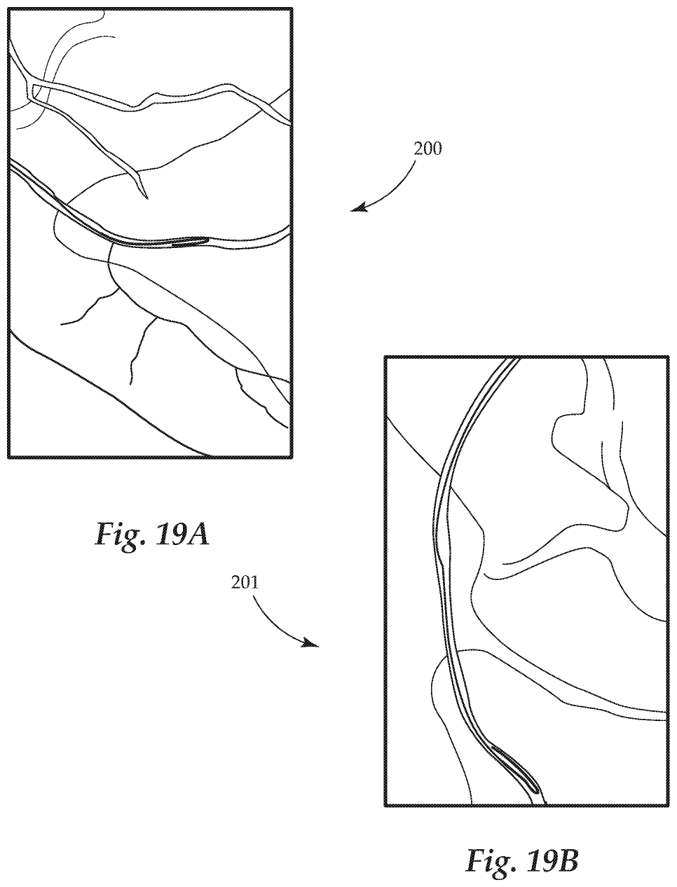

FIG. 19A-19B illustrate how one embodiment of the instant novel stent is designed to sustain high filling pressure to the microcirculation for better and faster wound healing. Further illustrated is the continued path of the tibio-pedal stent deployed from the later planar to the distal posterior tibial artery. FIG. 19A illustrates the continued phase of the release of the tibio-pedal stent 200 for one embodiment of the new specialty self-expanding stent deployed from the later planar to the distal posterior tibial artery. FIG. 19B illustrates the final fully extended phase of the release of the stent 201 for one embodiment of the for the new specialty self-expanding stent deployed from the later planar to the distal posterior tibial artery.

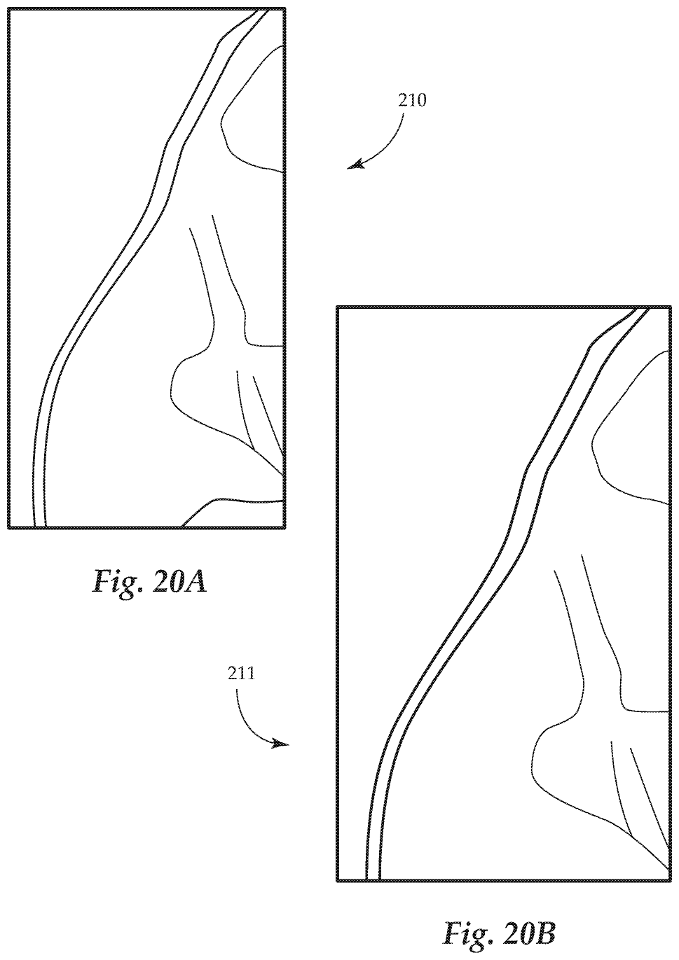

FIG. 20A-20B illustrate one embodiment of the stent mechanism and illustrate the flexibility of the stent mechanism within the system. FIG. 20A illustrates how the stent 210 conforms to the different arterial diameters, which met with the inherent design character required in the design process, in order to accommodate going from 1.5 mm to 4 mm if required. FIG. 20B illustrates a scenario wherein the stent 211 will dilate to the underline lumen or to the post dilated balloon. Additionally illustrated is the stent transitioning from the planar artery to posterior tibial artery PTA

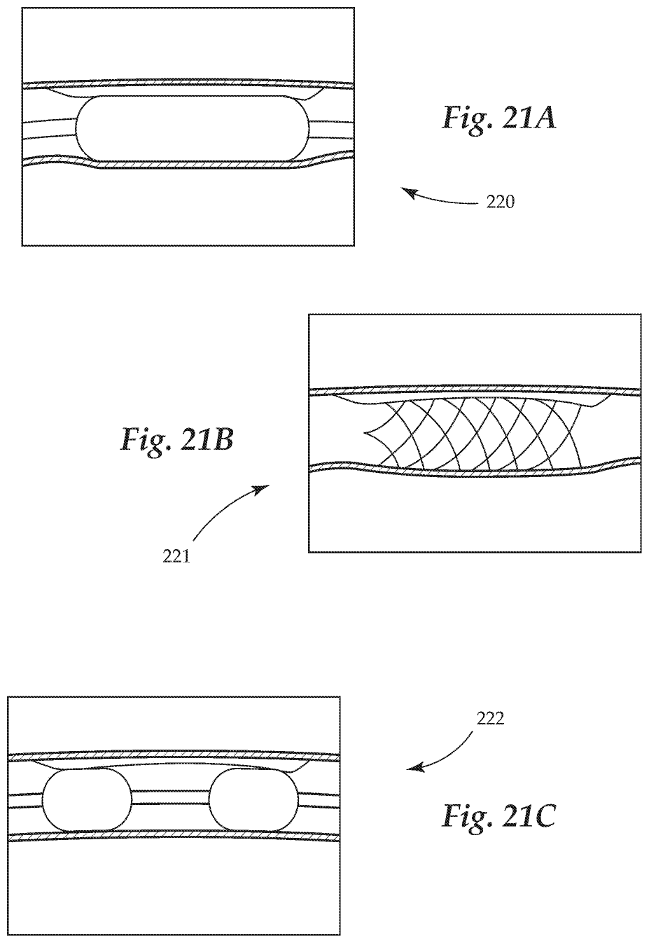

FIG. 21A-21C illustrate one embodiment of the stent mechanism in place and varying positions of Posterior Tibial Artery (PTA) balloons. FIG. 21A illustrates one position of a Posterior Tibial Artery (PTA) balloon 220. FIG. 21B illustrates a cross-section of an artery of the stent 221 in place. Moreover, FIG. 21C illustrates one position of a Posterior Tibial Artery (PTA) balloon 222.

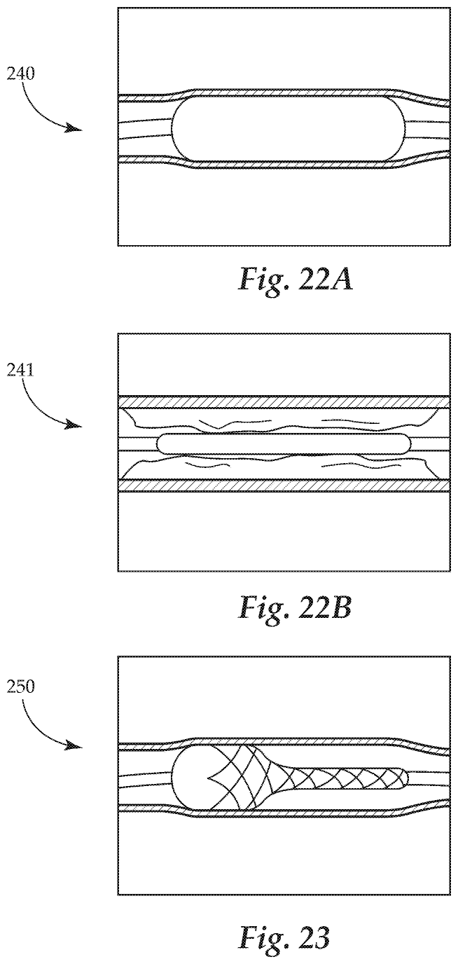

FIG. 22A-22B illustrate one embodiment of the stent mechanism in place and varying positions of Posterior Tibial Artery (PTA) balloons. FIG. 22A illustrates one position of a Posterior Tibial Artery (PTA) balloon 240. FIG. 22B illustrates a cross-section of an artery of the stent 241 in place. Also illustrated is the stent 241 transitioning from the planar artery to posterior tibial artery PTA. FIG. 23 illustrates the partially deployed stent 250 illustrating the immediate wall opposition.

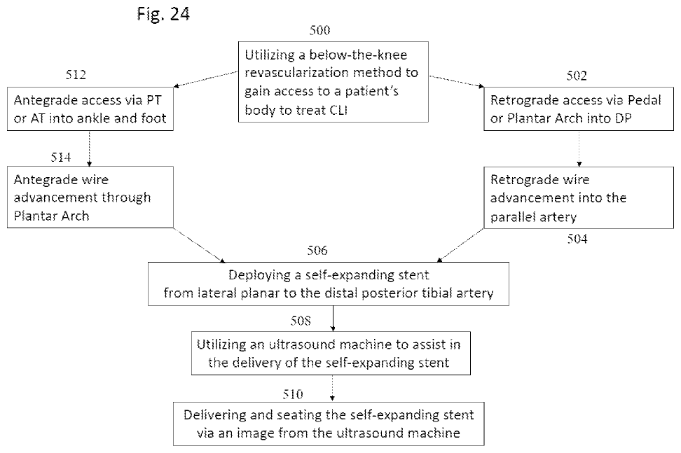

FIG. 24 illustrates a flow diagram for one embodiment of a method to treat critical limb ischemia by inserting a stent into a patient's body via the superficial femoral artery. In a preferred embodiment, at step 500, a below-the-knee revascularization method is utilized to gain access to a patient's body to treat CLI. In this embodiment, there are two access routes that may be used by a surgeon performing the procedure. At step 502, a retrograde access via either the pedal or plantar arch into the dorsalis pedis (a.k.a. dorsal artery of the foot). Next, at step 504, retrograde wire advancement is made into the parallel artery, and then a self-expanding stent is deployed from the lateral planar to the distal posterior tibial artery at step 506. Following the deployment of the stent, at step 508, an ultrasound machine is utilized to assist in the delivery of the self-expanding stent. Lastly, at step 510, the self-expanding stent is delivered and seated at a target location, wherein the ultrasound machine provides an image to the surgeon of the target location of the stent. Alternatively, at step 512, following utilization of the below-the-knee revascularization method at step 500, antegrade access is gained via the posterior tibial artery or anterior tibial artery into the ankle and foot. Then, at step 514, the guidewire of the stent is advanced antegrade through the plantar arch. At this point, step 506 through 510 are followed to deliver the self-expanding stent to the target location.

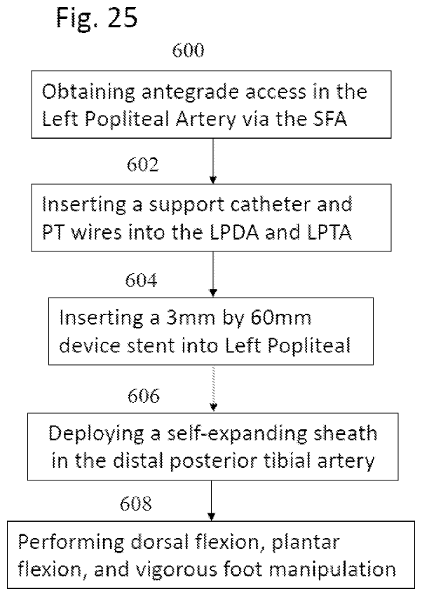

FIG. 25 illustrates a flow diagram of one embodiment to treat critical limb ischemia, wherein at step 600, antegrade access to the left popliteal artery is gained via the SFA. Following antegrade access, at step 602 a surgeon performing the operation inserts a support catheter and posterior tibial wires into the left posterior descending artery and the left posterior tibial artery. Next, at step 604, a stent (preferably a 3 mm by 60 mm) is inserted into the left popliteal artery. Once the stent is inserted at step 604, then a self-expanding sheath is deployed at step 606 in the distal posterior tibial artery. Lastly, at step 608, dorsal flexion, plantar flexion and vigorous foot manipulation is performed by the surgeon performing the operation.

In many prior art stent placement scenarios, mechanisms including a strand pre-loaded stent delivery system comprising an outer sheath, a compressed stent loaded therein, and a conventional stabilizer loaded adjacent to the proximal end of the stent. “Proximal” end refers to the end closer to an entry location outside the body. “Distal” end refers to the farthest end from the entry location. The term “stabilizer” is used in the art to describe component of stent delivery systems used to stabilize or prevent retraction of stent when sheath is retracted, thus effecting deployment of the stent into a desired location. The stabilizer limits the movement between the sheath and the stent in order to provide accurate and precise placement.

Additionally, a delivery system comprising a catheter tip as its distal end, which is attached to an internal sheath that runs through the delivery system through the inner lumen in stabilizer. A stabilizer handle is typically located at the proximal end of the stabilizer, outside the body lumen.

To position and deploy the stent, delivery system is directed through the body lumen to the patient's desired and needed location for stent deployment and vessel repair. Outer sheath is then retracted, and stabilizer acts as a stabilizer to keep stent from retracting with the sheath. As outer sheath retracts, stent is exposed and expands into place in the patient's body.

In one embodiment, to position and deploy the stent, a delivery system is directed through the body lumen to the patient's desired and needed location for stent deployment and vessel repair. Outer sheath is then retracted, and stabilizer acts as a stabilizer to keep stent from retracting with the sheath. As outer sheath retracts, stent is exposed and expands into place in the patient's body.

In an additional one embodiment for a stent stabilizer and pusher mechanism that may be utilized in conjunction with and as a stent delivery system. In one embodiment, the stent stabilizer and pusher mechanism may comprise a reinforced polymer shaft, wherein the polymer shaft may be constructed to have low friction. The polymer shaft further comprises a lumen; in one embodiment the low friction lumen may be guide wire compatible. The polymer shaft may further comprise a proximal end and a distal end, wherein the proximal end may be defined as meaning closer to an entry location outside the body. In one embodiment, a pusher hub is positioned at the proximal end of the shaft, and a braided mesh sock is positioned at the distal end of the shaft.

In an additional embodiment, the braided mesh sock may be constructed with 36 strands of 0.001″ nickel titanium. Furthermore, in another embodiment, the braided mesh sock would comprise a construction of one over braid and one under braid with a braid angle at approximately one hundred twenty degrees. In separate embodiments, the braid configuration and braid material for the braided mesh sock may comprise any braid and material, which may successfully capture and pull a stent inside a corresponding guide wire. In one embodiment, the braided mesh sock is constrained at the distal end of the shaft beneath a marker band.

Additionally, the shaft may comprise an inner diameter and an outer diameter, wherein the inner diameter of the shaft is approximately 0.021″ and the outer diameter of the shaft is approximately 0.030″. Furthermore, the mesh sock may include a diameter which is a heat set diameter and is approximately 0.5 mm. In one embodiment, it is preferable that the heat set diameter be approximately 0.5-1.0 mm larger than the stent captured by the braided mesh sock.

In an additional embodiment may include partial cross-sectional of the stent delivery system showing the stent stabilizer and pusher mechanism engaging a braided stent and located within a catheter. In this embodiment, the catheter comprises a polymer shaft with a proximal end and a distal end.

In this embodiment, the braided mesh sock attached to the outer diameter of the stent stabilizer and pusher mechanism, within the catheter. Furthermore, the braided mesh sock is bonded to the marker band, and the bond is terminated beneath the marker band.

Additionally, the braided mesh sock may be engaged with the braided stent. In one embodiment, the stabilizer and pusher mechanism may accept a guide wire through the lumen, which assists with the tracking of the stent delivery system. At the proximal end of the catheter there may be a catheter hub.

The stent delivery system may feature loading the braided stent inside a 3 French (“3 F”) catheter using the stent stabilizer and pusher mechanism. At the distal end of the catheter the braided mesh sock is collapsed and secured around the braided stent. In one embodiment, the stent mechanism is held butted against an inside of the mesh sock. Also, the mesh sock may comprise a constrained end and an unconstrained end and the retraction of the stent stabilizer and pusher mechanism pulls the braided stent inside the catheter toward the hub as depicted by a directional arrow. In this embodiment the braided stent is pulled toward the proximal end of the catheter.

As illustrated in FIG. 5, gaining access may occur in several manners, including but not limited to:

Antegrade Access via PT or AT into ankle and foot

Retrograde access via Pedal or Plantar Arch into DP

Micropuncture kits are very useful for pedal access:
Cook Micropuncture Access Kit

There may be numerous embodiments of the radiopaque stent delivery system, utilizing various diameters and ranges.

In some embodiments, a range of rations of platinum to nitinol may be employed, including rations of 5% to 50%. However, the utilization of 10% Platinum to Nitinol ration represents an optimum compromise between visualization and mechanical performances as every increase in platinum, the wire becomes more visible but the material properties diminish, which makes the stent weaker and less flexible.

In a preferred embodiment, the radiopaque stent includes the platinum element wherein the atomic percent is greater than or equal to 2.5 and less than or equal to 15. With these characteristics, the platinum still comprises high radiopacity but does not inhibit the flexibility and elastic qualities of the nitinol.

For prospective crossing and Support configurations, numerous Wires may be employed including hydrophilic wires may be utilized including Boston Scientific PT2 Support Guidewire, Abbott Hi-Torque Command Guidewire, Approach Hydro ST Microwire Guide.

Additionally, numerous catheters may be employed including a Spectranetics Quickcross Support Catheter and a Cook CXI Support Catheter.

Utilizing said components, the system enables below the knee entry and around the ankle penetration through the small arteries.

Future potential therapies will potentially allow below the ankle stenting. In one embodiment, a 3 F Balloon and stent compatible system visible by ultrasound may be introduced. With such low-profile devices, that are seen via DUS and allow for minimal bleeding, CLI patients will eventually be able to receive procedures in an office-based outpatient setting.

In one embodiment, a novel stenting system, set of devices and methodology are introduced. In practice, one embodiment features obtaining antegrade access in the Left Popliteal Artery with a 7 Fr. Sheath. Next, the doctor or technician may insert a Navicross (support catheter) and PT wires may also be inserted into left posterior descending artery (LPDA) and LPTA.

Subsequently, a 3 mm by 60 mm device stent may be inserted into Left Popliteal and a Self-expanding sheath may be deployed in the distal posterior tibial artery. Next, dorsal flexion, plantar flexion and vigorous manipulation of foot may be performed in order to show stent integrity.

Thus, in one embodiment, the system may be designed to install and utilize a stent located across the ankle joint. The stent may comprise the different embodiments to conform to the different arterial diameters and designed to accommodate going from 1.5 mm to 4 mm if needed in situations. In one embodiment, the stent will dilate to the underline lumen or to the post dilated balloon, transitioning from plantar artery to PT artery or posterior tibial artery of the lower limb carries blood to the posterior compartment of the leg and plantar surface of the foot, from the popliteal artery via the tibial-fibular trunk.

It is accompanied by a deep vein, the posterior tibial vein (“PT”), along its course. The PT gives rise to the medial plantar artery, lateral plantar artery, and gives rise to the fibular artery. Often, the branch of the fibular artery is said to rise from the bifurcation of the tibial-fibular trunk and the posterior tibial artery.

There may be numerous differing constructions or permutations of the system and the system is optimally designed for pedal access. To discuss at least four, in one embodiment, the system is designed for a 0.014 wire with two lengths, a short version utilizing a length of fifteen (15) centimeters and long version utilizing a length of 100 (100) centimeters. In an additional embodiment, the system may be designed for a 0.039 wire with two lengths, a short version utilizing a length of fifteen (15) centimeters and long version utilizing a length of 100 (100) centimeters.

In one embodiment, the system may possess an embarked stent for delivery to a proscribed area for a specific purpose such as treatment or assistance with vascular occlusion or other blockage of blood vessels. In one embodiment, the stent may utilize multipurpose beads which afford the stent the ability to stick to the wall.

In one embodiment, a 4 Fr. compatible stent (in one embodiment deemed a MicroStent™) used in conjunction with a one hundred and twenty centimeter (“120 cm”) delivery catheter, as well as accompanying stent devices disposed in five lengths and five diameters to meet individual patient needs. Specifically, the MicroStent™ is a vascular stent specifically designed to reduce below-the-knee amputations caused by critical limb ischemia (CLI) resulting from peripheral artery disease.

Peripheral artery disease can make it challenging to navigate small, calcified blood vessels in the lower leg. Using the instant 120 cm MicroStent™ catheter, physicians will now have multiple access points above the knee, expanding and optimizing the treatment options for CLI. This system enables below the knee entry and around the ankle penetration through the small arteries. Currently there are no favorable stents on the market meet the needs for below the ankle entry and around the ankle penetration.

In additional embodiments, MicroStent™ may include sizes of 8 mm, 15 mm, 25 mm, 40 mm and 60 mm will allow physicians to choose the stent length based on the best fit for each patient's diseased artery, allowing for a more customized approach. Furthermore, for patients with CLI, insufficient blood flow to the lower leg causes pain and, eventually, tissue damage. In the past, many instances would result in amputation below the knee. However, with the advent of the instant procedures and utilization of the MicroStent™, the vessel may be restored, pain may be alleviated, and the limb may be ultimately be saved.

Therefore, the advent of a one hundred and twenty centimeter one hundred and twenty centimeter (“120 cm” catheter provides more options and allows the physician or technician the capability to choose the best access point for the individual patient due to the added length for delivery. In an additional embodiment, a 3.2 Fr compatible stent used in conjunction with the one hundred and twenty centimeter (“120 cm”) delivery catheter is introduced. The robust 3.2 Fr shaft provides excellent push and control when transmitting movement from the more distant femoral access point and thus the perfect balance of femoral access and control.”

The multipurpose beads may also be utilized to provide vision assistance when utilizing placement detection apparatuses such as X-ray and ultrasound. This system may be utilized on a micro level for below the knee stenting purposes, particularly around the ankle
Disease State: Peripheral Artery Disease (PAD) Critical Limb Ischemia (CLI)
Therapeutic Area: Below The Knee (BTK)
Peripheral Artery Disease (PAD) and Critical Limb Ischemia (CLI)
   Affects 215 million people worldwide in 2015, predicted to be 230 million by 2020¹
   Problem continues to escalate due to the aging population, obesity, diabetes and sedentary life-style.
Atherosclerosis in the blood supply settles in the extremities reducing circulation
85% of amputations are due to poor circulation from Diabetes²
50% Mortality Rate 1 year after amputation³
No current stent solution for below the knee disease

What is claimed:

1. A method of treating critical limb ischemia (CLI) comprising the steps of:
obtaining antegrade access in the left popliteal artery with a 7 fr. sheath.
inserting a support catheter;
inserting a set of PT wires may also be inserted into left posterior descending (LPDA) artery and left posterior tibial (LPTA) artery.
inserting a stent device into the left popliteal and a self-expanding sheath may be deployed in the distal posterior tibial artery.
performing dorsal flexion;
performing plantar flexion;
manipulating the patient's foot vigorously in order to verify stent integrity.

2. The method of treating critical limb ischemia (CLI) of claim 1 wherein the support catheter comprises a Navicross® support catheter.

3. The method of treating critical limb ischemia (CLI) of claim 1 wherein the stent device comprises a 3 mm by 60 mm stent device.

4. The method of treating critical limb ischemia (CLI) of claim 1 further comprising the step of utilizing a 3 F Balloon.

5. The method of treating critical limb ischemia (CLI) of claim 1 further comprising the step of utilizing a 3 F Balloon and Stent compatible system visible by ultrasound.

6. The method of treating critical limb ischemia (CLI) of claim 1 further comprising the step of utilizing a 3 F Balloon and Stent compatible system visible by digital ultra sound (“DUS”).

7. The method of treating critical limb ischemia (CLI) of claim 1 wherein system may be designed to install and utilize a stent located across the ankle joint.

8. The method of treating critical limb ischemia (CLI) of claim 1 wherein the stent may comprise the different structures disposed to conform to arterial diameters in a range from 1.5 mm to 4 mm.

9. The method of treating critical limb ischemia (CLI) of claim 1 wherein a 4 Fr. compatible stent is utilized in conjunction with a one hundred and twenty centimeter (“120 cm”).

10. A method of treating critical limb ischemia (CLI) within the superficial femoral arteries SFA comprising the steps of:
obtaining antegrade access in the left popliteal artery with a 7 fr. sheath.
inserting a support catheter;
inserting a set of PT wires may also be inserted into left posterior descending (LPDA) artery and left posterior tibial (LPTA) artery.
inserting a stent device into the left popliteal;
traversing lateral plantar branch, into pedal arch, through dorsalis pedis, back into the anterior tibial artery in the anterior compartment of the leg;
deploying a self-expanding sheath may be deployed in the distal posterior tibial artery.
performing dorsal flexion;
performing plantar flexion;
manipulating the patient's foot vigorously in order to verify stent integrity.
